SEQUENCE LISTING (4 sequences)

SEQ ID NO: 1 - RNA, 75 nt, Artificial Sequence
  RNA template
ugugccggucuuucugaacgggauauaaaccuggccagcuucaucgaacaaguugccgugucuaugacauagauc

SEQ ID NO: 2 - DNA, 17 bp, Artificial Sequence
  PCR primer 5B.2F
atggacaggcgccctga

SEQ ID NO: 3 - DNA, 20 bp, Artificial Sequence
  PCR primer 5B.2R
ttgatgggcagcttggtttc

SEQ ID NO: 4 - DNA, 17 bp, Artificial Sequence
  FAM labeled probe
cacgccatgcgctgcgg

(19) United States
(12) Patent Application Publication
(10) Pub. No.: US 2013/0164259 A1
(43) Pub. Date: Jun. 27, 2013

(54) 1,4-SUBSTITUTED PIPERAZINE DERIVATIVES AND METHODS OF USE THEREOF

(71) Applicants: Cliff C. Cheng, Cambridge, MA (US); Xiaohua Huang, Malden, MA (US); Gerald W. Shipps, JR., Stoneham, MA (US)

(72) Inventors: Cliff C. Cheng, Cambridge, MA (US); Xiaohua Huang, Malden, MA (US); Gerald W. Shipps, JR., Stoneham, MA (US)

(21) Appl. No.: 13/518,317

(22) PCT Filed: Dec. 20, 2010
(86) PCT No.: PCT/US10/61203

§ 371 Date: Jun. 21, 2012

Related U.S. Application Data
(60) Provisional application No. 61/289,195, filed on Dec. 22, 2009.

(51) Int. Cl. A61K 31/497; C07D 403/04; C07D 409/14; C07D 471/04; C07D 413/14; C07D 405/14; C07D 403/14; A61K 31/501; A61K 31/506; A61K 31/496; C07D 417/14; C07D 401/12; C07D 495/04; A61K 31/519; C07D 487/04; A61K 31/7088; A61K 39/00; A61K 38/21; A61K 39/395; A61K 39/29; A61P 37/04; A61P 31/12; A61P 31/14; C07D 401/14

(52) U.S. Cl. 424/85.4; 544/357; 514/252.11; 544/295; 544/238; 514/252.02; 514/252.18; 514/253.1; 544/364; 544/365; 514/253.13; 544/278; 514/252.16; 544/280; 514/44 A; 424/184.1; 424/130.1; 424/141.1; 424/228.1

(57) ABSTRACT

The present invention relates to 1,4-Substituted Piperazine Derivatives, compositions comprising one or more 1,4-Substituted Piperazine Derivatives, and methods of using the 1,4-Substituted Piperazine Derivatives for treating or preventing a viral infection or a virus-related disorder in a patient.

### FIELD OF THE INVENTION

The present invention relates to 1,4-Substituted Piperazine Derivatives, compositions comprising one or more 1,4-Substituted Piperazine Derivatives, and methods of using the 1,4-Substituted Piperazine Derivatives for treating or preventing a viral infection or a virus-related disorder in a patient.

### BACKGROUND OF THE INVENTION

HCV is a (+)-sense single-stranded RNA virus that has been implicated as the major causative agent in non-A, non-B hepatitis (NANBH). NANBH is distinguished from other types of viral-induced liver disease, such as hepatitis A virus (HAV), hepatitis B virus (HBV), hepatitis delta virus (HDV), as well as from other forms of liver disease such as alcoholism and primary biliary cirrhosis.

Hepatitis C virus is a member of the hepacivirus genus in the family Flaviviridae. It is the major causative agent of non-A, non-B viral hepatitis and is the major cause of transfusion-associated hepatitis and accounts for a significant proportion of hepatitis cases worldwide. Although acute HCV infection is often asymptomatic, nearly 80% of cases resolve to chronic hepatitis. About 60% of patients develop liver disease with various clinical outcomes ranging from an asymptomatic carrier state to chronic active hepatitis and liver cirrhosis (occurring in about 20% of patients), which is strongly associated with the development of hepatocellular carcinoma (occurring in about 1-5% of patients). The World Health Organization estimates that 170 million people are chronically infected with HCV, with an estimated 4 million living in the United States.

HCV has been implicated in cirrhosis of the liver and in induction of hepatocellular carcinoma. The prognosis for patients suffering from HCV infection remains poor as HCV infection is more difficult to treat than other forms of hepatitis. Current data indicates a four-year survival rate of below 50% for patients suffering from cirrhosis and a five-year survival rate of below 30% for patients diagnosed with localized resectable hepatocellular carcinoma. Patients diagnosed with localized unresectable hepatocellular carcinoma fare even worse, having a five-year survival rate of less than 1%.

It is well-established that persistent infection of HCV is related to chronic hepatitis, and as such, inhibition of HCV replication is a viable strategy for the prevention of hepatocellular carcinoma. Present treatment approaches for HCV infection suffer from poor efficacy and unfavorable side-effects and there is currently a strong effort directed to the discovery of HCV replication inhibitors that are useful for the treatment and prevention of HCV related disorders. New approaches currently under investigation include the development of prophylactic and therapeutic vaccines, the identification of interferons with improved pharmacokinetic characteristics, and the discovery of agents designed to inhibit the function of three major viral proteins: protease, helicase and polymerase. In addition, the HCV RNA genome itself, particularly the IRES element, is being actively exploited as an antiviral target using antisense molecules and catalytic ribozymes.

Particular therapies for HCV infection include α-interferon monotherapy and combination therapy comprising α-interferon and ribavirin. These therapies have been shown to be effective in some patients with chronic HCV infection. The use of antisense oligonucleotides for treatment of HCV infection has also been proposed as has the use of free bile acids, such as ursodeoxycholic acid and chenodeoxycholic acid, and conjugated bile acids, such as tauroursodeoxycholic acid. Phosphonoformic acid esters have also been proposed as potentially for the treatment of various viral infections including HCV. Vaccine development, however, has been hampered by the high degree of viral strain heterogeneity and immune evasion and the lack of protection against reinfection, even with the same inoculum.

The development of small-molecule inhibitors directed against specific viral targets has become a major focus of anti-HCV research. The determination of crystal structures for NS3 protease, NS3 RNA helicase, and NS5B polymerase, with and without bound ligands, has provided important structural insights useful for the rational design of specific inhibitors.

NS5B, the RNA-dependent RNA polymerase, is an important and attractive target for small-molecule inhibitors. Studies with pestiviruses have shown that the small molecule compound VP32947 (3-[((2-dipropylamino)ethyl)thio]-5H-1,2,4-triazino[5,6-b]indole) is a potent inhibitor of pestivirus replication and most likely inhibits the NS5B enzyme since resistant strains are mutated in this gene. Inhibition of RdRp activity by (−)β-L-2′,3′-dideoxy-3′-thiacytidine 5′-triphosphate (3TC; lamivudine triphosphate) and phosphonoacetic acid also has been observed.

Despite the effort directed at the treatment and prevention of HCV and related viral infections, there exists a need in the art for non-peptide, small-molecule compounds having desirable or improved physicochemical properties that are useful for inhibiting viruses and treating viral infections and virus-related disorders.

### SUMMARY OF THE INVENTION

In one aspect, the present invention provides compounds having the formula:

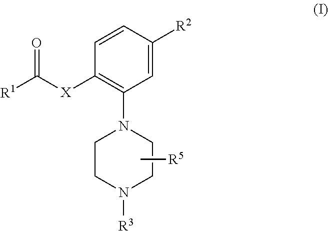

and pharmaceutically acceptable salts thereof,
wherein:

X is —O—, or —N(alkyl)-;

R¹ is aryl, cycloalkyl, heteroaryl or heterocycloalkyl, wherein said aryl group, said cycloalkyl group, said heteroaryl group or said heterocycloalkyl group can be optionally substituted with up to 3 substituents, which can be the same or different, and are selected from alkyl, alkenyl, alkynyl, cycloalkyl, aryl, halo, haloalkyl, —N(R⁴)(R⁴), —OR⁴, —S(O)₂-alkyl, —NHC(O)R⁴, —C(O)N(R⁴)(R⁴) or —C(O)OR⁴;

R² is alkyl, halo, haloalkyl, alkenyl, alkynyl, cycloalkyl, aryl, heteroaryl, heterocycloalkyl, —C(O)OR⁴, —N(R⁴)(R⁴), —OR⁴, —S(O)₂-alkyl, —NHC(O)R⁴ or —C(O)N(R⁴)(R⁴);

R³ is aryl, heteroaryl, —S(O)₂-alkyl, —C(O)N(R⁴)(R⁴), —C(O)-alkylene-N(R⁴)(R⁴) or —C(O)OR⁴, wherein said aryl group or said heteroaryl group can be optionally substituted with up to 3 substituents, which can be the same or different, and are selected from alkyl, aryl, cycloalkyl, heterocycloalkyl, heteroaryl, halo, -alkylene-S-alkyl, alkenyl, alkynyl, —N(R⁴)(R⁴), —S(O)₂-alkyl, —NHC(O)R⁴, —C(O)N(R⁴)(R⁴) or —C(O)OR⁴;

each occurrence of R⁴ is independently H, alkyl, aryl, cycloalkyl, heterocycloalkyl or heteroaryl; and

R⁵ represents one or more optional piperazine ring carbon substituents, which can be the same or different, and are selected from alkyl, cycloalkyl, halo, haloalkyl, —N(R⁴)(R⁴), —OR⁴, —S(O)₂-alkyl, —NHC(O)R⁴, —C(O)N(R⁴)(R⁴) or —C(O)OR⁴.

The Compounds of Formula (I) (also referred to herein as the “1,4-Substituted Piperazine Derivatives”) and pharmaceutically acceptable salts thereof can be useful for treating or preventing a viral infection in a patient.

The Compounds of Formula (I) (also referred to herein as the “1,4-Substituted Piperazine Derivatives”) or pharmaceutically acceptable salts, solvates, prodrugs or esters thereof can also be useful for treating or preventing a virus-related disorder in a patient.

Also provided by the invention are methods for treating or preventing a viral infection or a virus-related disorder in a patient, comprising administering to the patient an effective amount of one or more 1,4-Substituted Piperazine Derivatives.

The present invention further provides pharmaceutical compositions comprising an effective amount of one or more 1,4-Substituted Piperazine Derivatives or a pharmaceutically acceptable salt thereof, and a pharmaceutically acceptable carrier. The compositions can be useful for treating or preventing a viral infection or a virus-related disorder in a patient.

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

The present invention provides 1,4-Substituted Piperazine Derivatives, pharmaceutical compositions comprising one or more 1,4-Substituted Piperazine Derivatives, and methods of using the 1,4-Substituted Piperazine Derivatives for treating or preventing a viral infection or a virus-related disorder in a patient.

### Definitions and Abbreviations

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “aminoalkyl,” “haloalkyl,” “alkoxy,” etc. . . .

As used herein, and throughout this disclosure, the following terms, unless otherwise indicated, shall be understood to have the following meanings:

A “patient” is a human or non-human mammal. In one embodiment, a patient is a human. In another embodiment, a patient is a chimpanzee.

The term “alkyl” as used herein, refers to an aliphatic hydrocarbon group, wherein one of the aliphatic hydrocarbon group's hydrogen atoms is replaced with a single bond.

An alkyl group can be straight or branched and can contain from about 1 to about 20 carbon atoms. In one embodiment, an alkyl group contains from about 1 to about 12 carbon atoms. In another embodiment, an alkyl group contains from about 1 to about 6 carbon atoms. Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or optionally substituted by one or more substituents which may be the same or different, each substituent being independently selected from the group consisting of halo, alkenyl, alkynyl, —O-aryl, aryl, heteroaryl, cycloalkyl, cycloalkenyl, cyano, —OH, —O-alkyl, —O-haloalkyl, -alkylene-O-alkyl, alkylthio, —NH₂, —NH(alkyl), —N(alkyl)₂, —NH-aryl, —NH-heteroaryl, —NHC(O)-alkyl, —NHC(O)NH-alkyl, —NHSO₂-alkyl, —NHSO₂-aryl, —NHSO₂-heteroaryl, —NH(cycloalkyl), —OC(O)-alkyl, —OC(O)-aryl, —OC(O)-cycloalkyl, —C(O)alkyl, —C(O)NH₂, —C(O)NH-alkyl, —C(O)OH and —C(O)O-alkyl. In one embodiment, an alkyl group is unsubstituted. In another embodiment, an alkyl group is a straight chain alkyl group. In another embodiment, an alkyl group is a branched alkyl group.

The term “aminoalkyl” as used herein, refers to an alkyl group, as defined above, wherein at least one of the alkyl group's hydrogen atoms is replaced with a group having the formula —N(R′)₂, wherein each occurrence of R′ is independently selected from H and alkyl. In one embodiment, an aminoalkyl group's alkyl moiety is linear. In another embodiment, an aminoalkyl group's alkyl moiety is branched. Illustrative examples of aminoalkyl groups include, but are not limited to, —CH₂CH₂NH₂, —CH₂CH(NH₂)CH₃, —CH₂CH₂CH₂NH₂, —CH₂CH₂NHCH₃, —CH₂CH₂N(CH₃)₂, —CH₂CH(N(CH₃)₂)CH₃ and —CH₂CH₂CH₂N(CH₃)₂.

The term “alkylene” as used herein, refers to an alkyl group, as defined above, wherein one of the alkyl group's hydrogen atoms is replaced with a bond. Illustrative examples of alkylene include, but are not limited to, —CH₂—, —CH₂CH₂—, —CH₂CH₂CH₂—, —CH₂CH₂CH₂CH₂—, —CH(CH₃)CH₂CH₂—, —CH₂CH(CH₃)CH₂— and —CH₂CH₂CH(CH₃)—. In one embodiment, an alkylene group is a straight chain alkylene group. In another embodiment, an alkylene group is a branched alkylene group.

The term “nitrogen-containing heteroaryl” as used herein, refers to an aromatic monocyclic or multicyclic ring system comprising about 5 to about 14 ring atoms, wherein one of the ring atoms is nitrogen, up to 3 remaining ring atoms can independently O, N or S, and the remaining ring atoms are carbon atoms. In one embodiment, a nitrogen-containing heteroaryl group has 5 to 10 ring atoms. In another embodiment, a nitrogen-containing heteroaryl group is monocyclic and has 5 or 6 ring atoms. In another embodiment, a nitrogen-containing heteroaryl group is bicyclic and has 9 or 10 ring atoms. A nitrogen-containing heteroaryl group can be joined via a ring carbon or ring nitrogen atom. A nitrogen or sulfur atom of a nitrogen-containing heteroaryl group can be optionally oxidized to the corresponding N-oxide, S-oxide or S,S-dioxide. The term “nitrogen-containing heteroaryl” also encompasses a nitrogen-containing heteroaryl group, as defined above, which has been fused to a benzene ring. Non-limiting examples of illustrative nitrogen-containing heteroaryls include pyridyl, pyrazinyl, pyrimidinyl, isoxazolyl, isothiazolyl, oxazolyl, thiazolyl, pyrazolyl, indazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, indolyl, azaindolyl, benzimidazolyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “nitrogen-containing heteroaryl” also refers to partially saturated nitrogen-containing multicyclic heteroaryl moieties such as, for example, tetrahydroisoquinolyl, tetrahydroquinolyl and the like. In one embodiment, a nitrogen-containing heteroaryl group is a 6-membered monocyclic nitrogen-containing heteroaryl group. In another embodiment, a nitrogen-containing heteroaryl group is a 5-membered monocyclic nitrogen-containing heteroaryl group. In another embodiment, a nitrogen-containing heteroaryl group is a 9-membered bicyclic nitrogen-containing heteroaryl group. In another embodiment, a nitrogen-containing heteroaryl group is a 10-membered bicyclic nitrogen-containing heteroaryl group.

The term “nitrogen-containing heterocycloalkyl” as used herein, refers to a non-aromatic saturated monocyclic or multicyclic ring system comprising 3 to about 10 ring atoms, wherein one of the ring atoms is nitrogen, up to 3 remaining ring atoms can independently O, N or S, and the remaining ring atoms are carbon atoms. In one embodiment, a nitrogen-containing heterocycloalkyl group has from about 5 to about 10 ring atoms. In another embodiment, a nitrogen-containing heterocycloalkyl group has 5 or 6 ring atoms. There are no adjacent oxygen and/or sulfur atoms present in the ring system. Any —NH group in a nitrogen-containing heterocycloalkyl ring may exist protected such as, for example, as an —N(Boc), —N(CBz), —N(Tos) group and the like; such protected nitrogen-containing heterocycloalkyl groups are considered part of this invention. The nitrogen or sulfur atom of a nitrogen-containing heterocycloalkyl group can be optionally oxidized to the corresponding N-oxide, S-oxide or S,S-dioxide. Non-limiting examples of illustrative monocyclic nitrogen-containing heterocycloalkyl rings include piperidyl, pyrrolidinyl, piperazinyl, morpholinyl, thiomorpholinyl, thiazolidinyl, lactam, and the like. A ring carbon atom of a nitrogen-containing heterocycloalkyl group may be functionalized as a carbonyl group. An illustrative example of such a nitrogen-containing heterocycloalkyl group is is pyrrolidonyl:

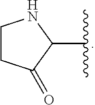 [SMILES: *C1NCCC1=O]

In one embodiment, a nitrogen-containing heterocycloalkyl group is a monocyclic 6-membered nitrogen-containing monocyclic nitrogen-containing heterocycloalkyl group. In another embodiment, a nitrogen-containing heterocycloalkyl group is a 5-membered monocyclic nitrogen-containing heterocycloalkyl group. In another embodiment, a nitrogen-containing heterocycloalkyl group is a 9-membered bicyclic nitrogen-containing heterocycloalkyl group. In another embodiment, a nitrogen-containing heterocycloalkyl group is a 10-membered bicyclic nitrogen-containing heterocycloalkyl group.

The term “nitrogen-containing heterocycloalkenyl” as used herein, refers to a nitrogen-containing heterocycloalkyl group, as defined above, wherein the nitrogen-containing heterocycloalkyl group contains from 3 to 10 ring atoms, and at least one endocyclic carbon-carbon or carbon-nitrogen double bond. In one embodiment, a nitrogen-containing heterocycloalkenyl group is bicyclic and has from 5 to 10 ring atoms. In another embodiment, a nitrogen-containing heterocycloalkenyl group is monocyclic and has 5 or 6 ring atoms. A nitrogen or sulfur atom of the nitrogen-containing heterocycloalkenyl can be optionally oxidized to the corresponding N-oxide, S-oxide or S,S-dioxide. Non-limiting examples of illustrative nitrogen-containing heterocycloalkenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, pyridone, 2-pyridone, dihydrothiopyranyl, and the like. A ring carbon atom of a nitrogen-containing heterocycloalkenyl group may be functionalized as a carbonyl group. An illustrative example of such a nitrogen-containing heterocycloalkenyl group is:

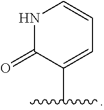 [SMILES: *c1ccc[nH]c1=O]

In one embodiment, a nitrogen-containing heterocycloalkenyl group is a 6-membered nitrogen-containing monocyclic heterocycloalkenyl group. In another embodiment, a nitrogen-containing heterocycloalkenyl group is a 5-membered nitrogen-containing monocyclic heterocycloalkenyl group. In another embodiment, a nitrogen-containing heterocycloalkenyl group is a 9-membered bicyclic nitrogen-containing heterocycloalkenyl group. In another embodiment, a nitrogen-containing heterocycloalkenyl group is a 10-membered nitrogen-containing bicyclic heterocycloalkenyl group.

The term “substituted,” as used herein, means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound' or “stable structure” is meant a compound that is sufficiently robust to survive isolation to a useful degree of purity from a reaction mixture, and formulation into an efficacious therapeutic agent.

The term “optionally substituted” as used herein, means optional substitution with the specified groups, radicals or moieties.

The terms “purified”, “in purified form” or “in isolated and purified form” as used herein, for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” as used herein, refers to a compound (e.g., a drug precursor) that is transformed in vivo to provide a 1,4-Substituted Piperazine Derivative or a pharmaceutically acceptable salt, hydrate or solvate thereof. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a 1,4-Substituted Piperazine Derivative or a pharmaceutically acceptable salt, hydrate or solvate of the compound contains a carboxylic acid functional group, a prodrug can comprise an ester fanned by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)aminoalkyl(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a 1,4-Substituted Piperazine Derivative contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)2, —P(O)(O(C₁-C₆)alkyl)₂ or glycosyl (the radical resulting from the removal of a hydroxyl group of the hemiacetal form of a carbohydrate), and the like.

If a 1,4-Substituted Piperazine Derivative incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)aminoalkylalkyl, —C(Y⁴)Y⁵ wherein Y⁴ is H or methyl and Y⁵ is mono-N— or di-N,N—(C₁-C₆)aminoalkyl morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of illustrative solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

The term “effective amount” as used herein, refers to an amount of 1,4-Substituted Piperazine Derivative and/or an additional therapeutic agent, or a composition thereof that is effective in producing the desired therapeutic, ameliorative, inhibitory or preventative effect when administered to a patient suffering from a viral infection or virus-related disorder. In the combination therapies of the present invention, an effective amount can refer to each individual agent or to the combination as a whole, wherein the amounts of all agents administered are together effective, but wherein the component agent of the combination may not be present individually in an effective amount.

Metabolic conjugates, such as glucuronides and sulfates which can undergo reversible conversion to the 1,4-Substituted Piperazine Derivatives are contemplated in the present invention.

The 1,4-Substituted Piperazine Derivatives may form salts, and all such salts are contemplated within the scope of this invention. Reference to a 1,4-Substituted Piperazine Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a 1,4-Substituted Piperazine Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a 1,4-Substituted Piperazine Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds which can be metabolically converted to the compounds to the present invention include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl-, aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) carboxylic acid esters obtained by esterification of a indole 2-carboxylic with a hydroxyl group of an alcohol, in which the alcohol is selected from from straight or branched chain alkylalcohols (for example, ethanol, n-propanol, t-butanol, or n-butanol), alkoxyalkanols (for example, methoxyethanol-), aminoalkanols (for example, aminoethanol, methylaminoethanol, dimethylaminoethanol, dimethylaminoprpanol), aralkanols (for example, benzyl alcohol), aryloxyalkanols (for example, phenoxymethanol), aryl alcohols (for example, phenol optionally substituted with, for example, halogen, C₁₋₄alkyl, or C₁₋₄alkoxy or amino); (3) sulfonate esters, such as alkyl- or aralkylsulfonyl (for example, methanesulfonyl); (4) amino acid esters (for example, L-valyl or L-isoleucyl); (5) phosphonate esters and (6) mono-, di- or triphosphate esters. The phosphate esters may be further esterified by, for example, a C₁₋₂₀ alcohol or reactive derivative thereof, or by a 2,3-di (C₆₋₂₄)acyl glycerol.

The 1,4-Substituted Piperazine Derivatives may contain asymmetric or chiral centers, and, therefore, exist in different stereoisomeric forms. It is intended that all stereoisomeric forms of the 1,4-Substituted Piperazine Derivatives as well as mixtures thereof, including racemic mixtures, form part of the present invention. In addition, the present invention embraces all geometric and positional isomers. For example, if a 1,4-Substituted Piperazine Derivative incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the 1,4-Substituted Piperazine Derivatives may be atropisomers (e.g., substituted biaryls) and are considered as part of this invention. Enantiomers can also be separated by use of chiral HPLC column.

The straight line [symbol] as a bond generally indicates a mixture of, or either of, the possible isomers, non-limiting example(s) include, containing (R)- and (S)-stereochemistry. For example,

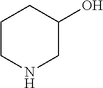

means containing both

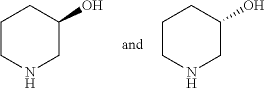 [SMILES: O[C@@H]1CCCNC1 ; O[C@H]1CCCNC1]

A dashed line ( [symbol] ) represents an optional and additional bond.

As well known in the art, a bond drawn from a particular atom wherein no moiety is depicted at the terminal end of the bond indicates a methyl group bound through that bond to the atom, unless stated otherwise. For example:

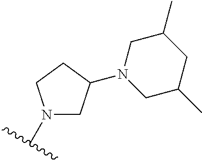 [SMILES: *N1CCC(N2CC(C)CC(C)C2)C1]

represents

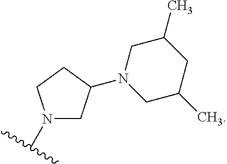 [SMILES: *N1CCC(N2CC(C)CC(C)C2)C1]

All stereoisomers (for example, geometric isomers, optical isomers and the like) of the present compounds (including those of the salts, solvates, hydrates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). For example, if a 1,4-Substituted Piperazine Derivative incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, positional isomers, racemates or prodrugs of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the 1,4-Substituted Piperazine Derivatives, and of the salts, solvates, hydrates, esters and prodrugs of the 1,4-Substituted Piperazine Derivatives, are intended to be included in the present invention.

The following abbreviations are used below and have the following meanings: DIEA is diisopropylethylamine, DMF is dimethylformamide, Et₃N is triethylamine, EtOAc is ethyl acetate, HATU is N-(diethylamino)-1H-1,2,3-triazolo[4,5-b]pyridine-1-yl-methylene]-N-methylmethanaminium hexafluorophosphate N-oxide, HPLC is high performance liquid chromatography, MeOH is methanol, Pd₂(dba)₃ is tris(dibenzylideneacetone)dipalladium(0), RuPhos is 2-dicyclohexylphosphino-2′,6′-diisopropoxy-1,1′-biphenyl, SPhos is 2-dicyclohexylphosphino-2′,6′-dimethoxybiphenyl, XPhos is 2-dicyclohexylphosphino-2′,4′,6′-triisopropylbiphenyl, THF is tetrahydrofuran, and TFA is trifluoroacetic acid.

### The Compounds of Formula (I)

The present invention provides 1,4-Substituted Piperazine Derivatives of Formula (I):

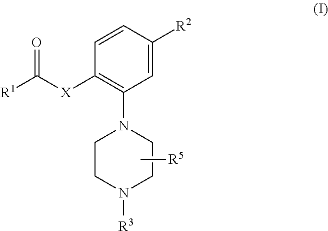

and pharmaceutically acceptable salts thereof, wherein R¹, R², R³ and R⁵ are defined above.

In one embodiment, X is —O—.

In another embodiment, X is —NH—.

In another embodiment, X is or —N(alkyl)-.

In one embodiment, R¹ is phenyl, heteroaryl or heterocycloalkyl.

In another embodiment, R¹ is aryl.

In another embodiment, R¹ is cycloalkyl.

In another embodiment, R¹ is heteroaryl.

In still another embodiment, R¹ is heterocycloalkyl.

In another embodiment, R¹ is pyridyl.

In another embodiment, R¹ is phenyl.

In yet another embodiment, R¹ is thiazolyl.

In another embodiment, R¹ is quinolinyl.

In a further embodiment, R¹ is thiophenyl.

In another embodiment, R¹ is furanyl.

In another embodiment, R¹ is 2-methyl pyridyl.

In still another embodiment, R¹ is 3,5-difluorobenzoyl.

In another embodiment, R¹ is:

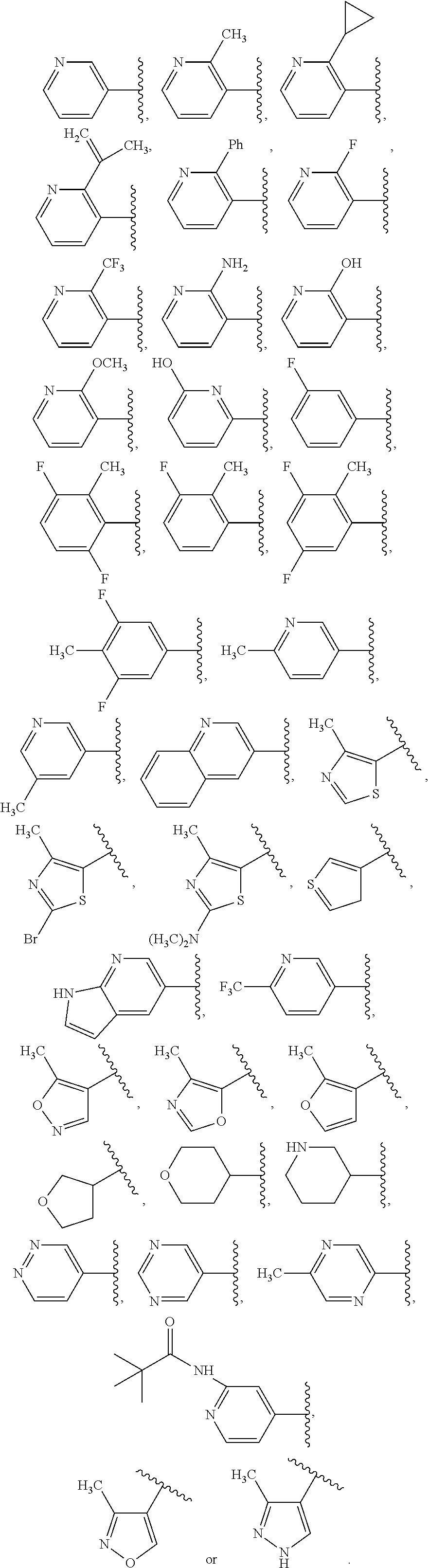

In one embodiment, R² is alkyl.

In another embodiment, R² is halo.

In another embodiment, R² is haloalkyl.

In still another embodiment, R² is alkenyl.

In another embodiment, R² is alkynyl.

In another embodiment, R² is cycloalkyl.

In yet another embodiment, R² is aryl.

In another embodiment, R² is heteroaryl.

In a further embodiment, R² is heterocycloalkyl.

In another embodiment, R² is —C(O)OR⁴.

In another embodiment, R² is —C(O)O-alkyl.

In still another embodiment, R² is —N(R⁴)(R⁴).

In another embodiment, R² is —OR⁴.

In another embodiment, R² is —S(O)₂-alkyl.

In yet another embodiment, R² is —NHC(O)R⁴.

In another embodiment, R² is —C(O)N(R⁴)(R⁴).

In one embodiment, R² is isopropyl.

In another embodiment, R² is t-butyl.

In another embodiment, R² is methyl.

In still another embodiment, R² is —C(CH₃)═CH

In another embodiment, R² is Br or I.

In another embodiment, R² is cyclopropyl.

In yet another embodiment, R² is phenyl.

In another embodiment, R² is —CF₃.

In a further embodiment, R² is —C(O)OCH₂CH₃.

In one embodiment, R³ is aryl.

In another embodiment, R³ is heteroaryl.

In another embodiment, R³ is 5-membered heteroaryl.

In still another embodiment, R³ is 6-membered heteroaryl.

In another embodiment, R³ is bicyclic heteroaryl.

In another embodiment, R³ is —S(O)₂-alkyl.

In yet another embodiment, R³ is —C(O)N(R⁴)(R⁴).

In another embodiment, R³ is —C(O)NH-phenyl.

In another embodiment, R³ is —C(O)-alkylene-N(R⁴)(R⁴).

In an further embodiment, R³ is —C(O)CH₂NHCH₃.

In another embodiment, R³ is —C(O)CH₂N(CH₃)₂.

In another embodiment, R³ is —C(O)OR⁴.

In still another embodiment, R³ is —C(O)O-alkyl.

In another embodiment, R³ is —C(O)O-t-butyl.

In another embodiment, R³ is thiazenopyrimidine or pyrrolopyrimidine.

In another embodiment, R³ is thiazenopyrimidine.

In yet another embodiment, R³ is pyrrolopyrimidine.

In one embodiment, R³ is:

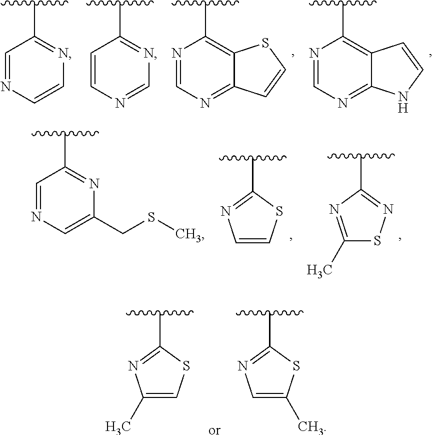

In another embodiment, R³ is:

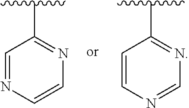 [SMILES: *c1cnccn1 ; *c1ccncn1]

In one embodiment, R⁵ is alkyl.

In another embodiment, R⁵ is cycloalkyl.

In another embodiment, R⁵ is halo.

In still another embodiment, R⁵ is haloalkyl.

In another embodiment, R⁵ is —N(R⁴)(R⁴).

In another embodiment, R⁵ is —OR⁴.

In yet another embodiment, R⁵ is —S(O)₂-alkyl.

In another embodiment, R⁵ is —NHC(O)R⁴.

In another embodiment, R⁵ is —C(O)N(R⁴)(R⁴).

In a farther embodiment, R⁵ is —C(O)OR⁴.

In another embodiment R⁵ is absent.

In one embodiment, R¹ is 2-methyl pyridyl or 3,5-difluorobenzoyl, and R³ is:

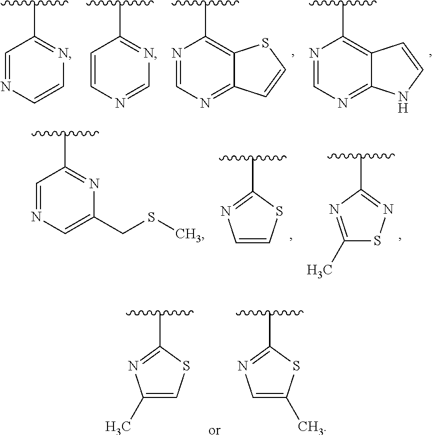

In one embodiment, one or more hydrogen atoms of a Compound of Formula (I) is replaced with a deuterium atom.

In another embodiment, for the Compounds of Formula (I), variables R¹, R², R³ and R⁵ are selected independently from each other.

In another embodiment, a Compound of Formula (I) is in purified form.

In one embodiment, a Compound of Formula (I) has the formula (Ia):

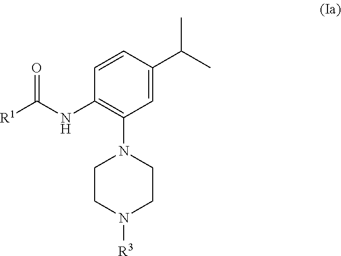

or a pharmaceutically acceptable salt thereof,
wherein:

R¹ is phenyl or pyridyl, wherein said phenyl group or said pyridyl group can be optionally substituted with up to 3 substituents, which can be the same or different, and are selected from alkyl, alkenyl, alkynyl, cycloalkyl, aryl, halo, haloalkyl, —N(R⁴)(R⁴), —S(O)₂-alkyl, —NHC(O)R⁴, —C(O)N(R⁴)(R⁴) or —C(O)OR⁴;

R³ is phenyl, 5- or 6-membered heteroaryl, or —C(O)O-alkyl, wherein said phenyl group or said 5- or 6-membered heteroaryl group can be optionally substituted with up to 3 substituents, which can be the same or different, and are selected from alkyl, aryl, cycloalkyl, heterocycloalkyl, heteroaryl, halo, -alkylene-S-alkyl, alkenyl, alkynyl, —N(R⁴)(R⁴), —OR⁴, —S(O)₂-alkyl, —NHC(O)R⁴, —C(O)N(R⁴)(R⁴) or —C(O)OR⁴; and

each occurrence of R⁴ is independently H, alkyl, aryl, cycloalkyl, heterocycloalkyl or heteroaryl.

In one embodiment, for the Compounds of Formula (Ia), R¹ is:

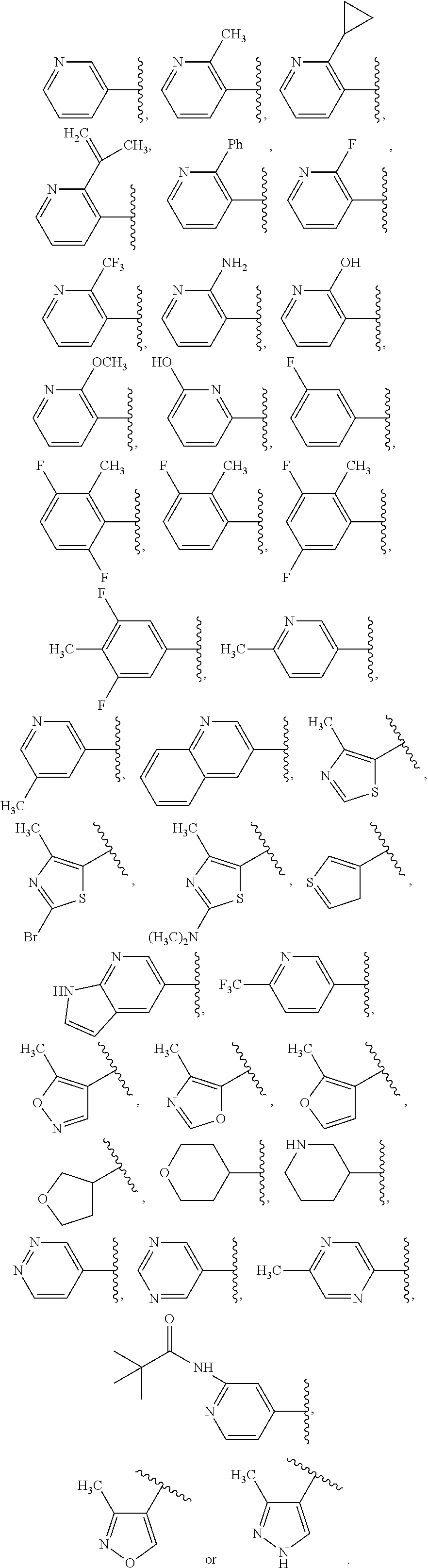

In another embodiment, for the Compounds of Formula (Ia), R³ is heteroaryl.

In another embodiment, for the Compounds of Formula (Ia), R³ is thiazenopyrimidine or pyrrolopyrimidine.

In still another embodiment, for the Compounds of Formula (Ia), R³ is:

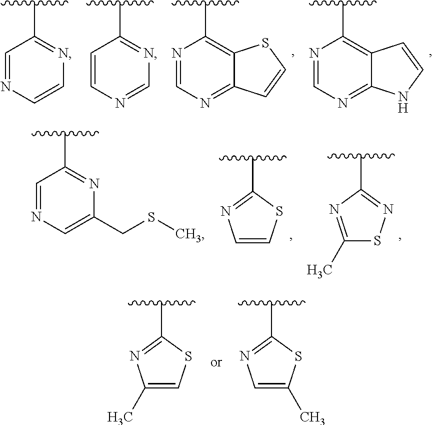

In another embodiment, for the Compounds of Formula (Ia), R¹ is:

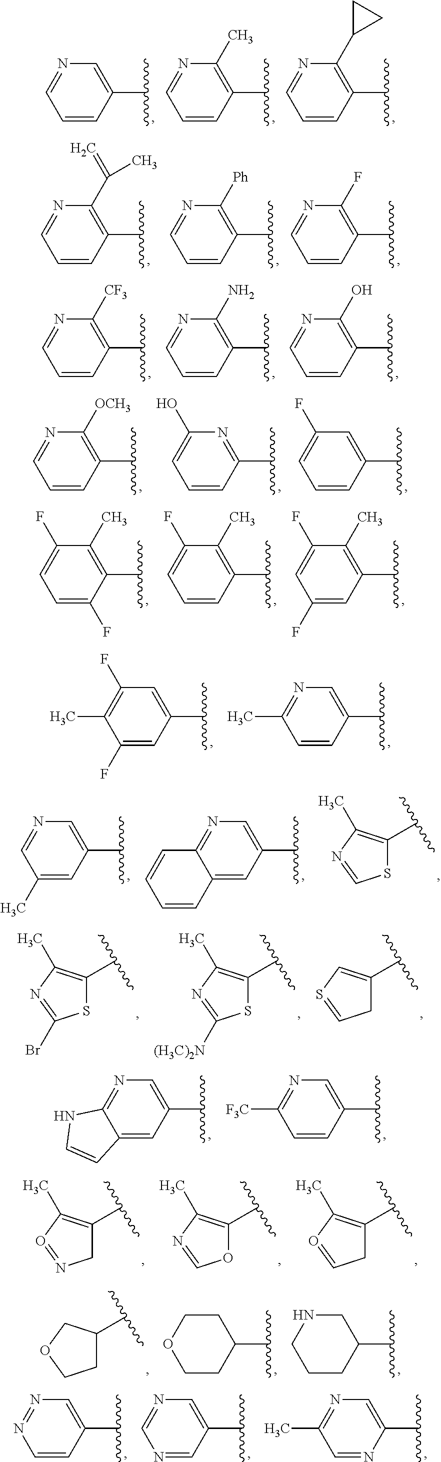

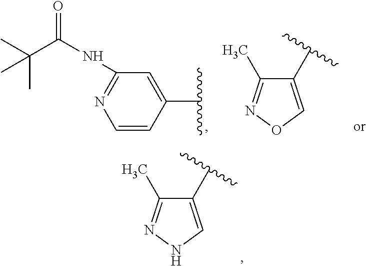

### and R³ is:

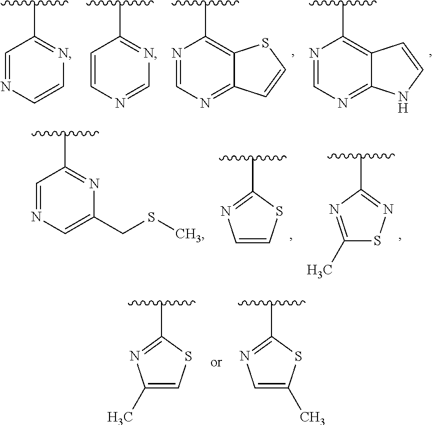

In one embodiment, one or more hydrogen atoms of a Compound of Formula (la) is replaced with a deuterium atom.

In another embodiment, for the Compounds of Formula (Ia), variables R¹ and R³ are selected independently of each other.

In another embodiment, a Compound of Formula (Ia) is in purified form.

Non-limiting examples of the Compounds of Formula (I) include compounds 1-70 as set forth in the Examples below, and pharmaceutically acceptable salts thereof.

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes 1 and 2. Alternative synthetic pathways and analogous structures will be apparent to those skilled in the art of organic synthesis. All stereoisomers and tautomeric forms of the compounds are contemplated.

Scheme 1 shows a method useful for making the Compounds of Formula (I), wherein X is —NH—.

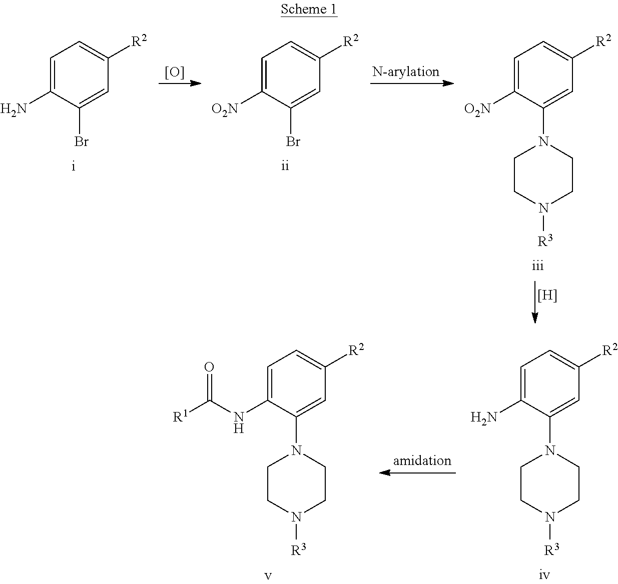

wherein R¹, R² and R³ are defined above for the Compounds of Formula (I).

A substituted bromoaniline of formula i can be oxidized to provide the corresponding nitro compounds of formula ii. A compound of formula ii can then be coupled with a 4-substituted piperizine compound to provide the phenyl piperazine compounds of formula iii. The nitro group of a compound of formula iii is then converted back to an amino group using standard methods to provide the compounds of formula iv. Finally, a compound of formula iv can be amidated using well-known methods to provide the compounds of formula v, which correspond to the Compounds of Formula (I), wherein X is —NH—.

Scheme 2 shows an alternative method useful for making the Compounds of Formula (I), wherein X is —NH— and R² is aryl or heteroaryl.

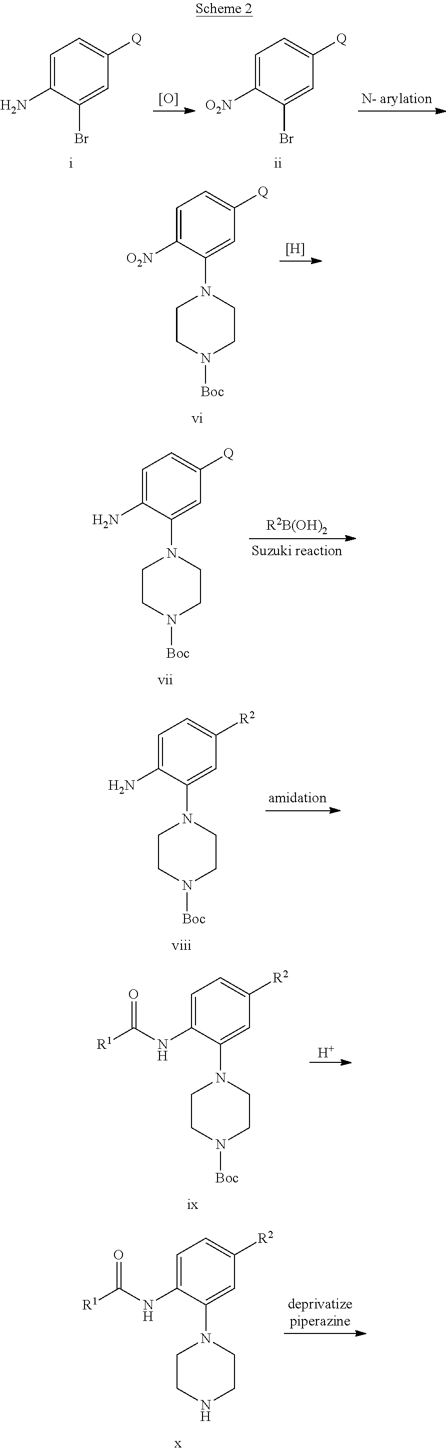

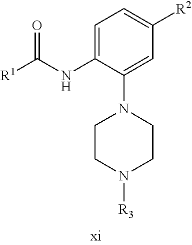

wherein R¹, R² and R³ are defined above for the Compounds of Formula (I).

A substituted bromoaniline of formula i can be oxidized to provide the corresponding nitro compounds of formula ii. A compound of formula ii can then be coupled with a 4-substituted piperizine compound to provide the phenyl piperazine compounds of formula vi. The nitro group of a compound of formula vi is then converted back to an amino group using standard methods to provide the compounds of formula vii. A compound of vii can then undergo a Suzuki coupling reaction with a substituted boronic acid of formula R²B(OH)₂, for example, to provide the compounds of formula viii, which has the aryl or heteroaryl R² group in place. A compound of formula viii can be amidated using well-known methods to provide the compounds of formula ix, which can subsequently be deprotected using acid to provide the piperizine compounds of formula x. Finally, the free piperazinyl nitrogen atom of a compound of formula x can then be derivatized using standard methods to install the R³ group and provide the compounds of formula xi, which which correspond to the Compounds of Formula (I), wherein X is —NH— and R² is aryl or heteroaryl.

The starting material and reagents depicted above are either available from commercial suppliers such as Sigma-Aldrich (St. Louis, Mo.) and Acros Organics Co. (Fair Lawn, N.J.), or can be prepared using methods well-known to those of skill in the art of organic synthesis.

One skilled in the art of organic synthesis will recognize that the synthesis of the core of the Compounds of Formula (I) may require the need for the protection of certain functional groups (i.e., derivatization for the purpose of chemical compatibility with a particular reaction condition). Suitable protecting groups for the various functional groups of these compounds and methods for their installation and removal can be found in Greene et al., Protective Groups in Organic Synthesis, Wiley-Interscience, New York, (1999).

One skilled in the art of organic synthesis will also recognize that one route for the synthesis of the core of the Compounds of Formula (I) may be more desirable than others, depending on the choice of appendage substituents. Additionally, one skilled in the art will recognize that in some cases the order of reactions may differ from that presented herein to avoid functional group incompatibilities and amend the synthetic route accordingly.

The preparation of ring systems contemplated in this invention have been described in the literature and in compendia such as “Comprehensive Heterocyclic Chemistry” editions I, II and III, published by Elsevier and edited by A. R. Katritzky & R J K Taylor. Manipulations of the required substitution patterns have also been described in the available chemical literature as summarized in compendia such as, for example, “Comprehensive Organic Chemistry” published by Elsevier and edited by D H R. Barton and W. D. Ollis; “Comprehensive Organic Functional Group Transformations” edited by edited by A. R. Katritzky & R J K Taylor; and “Comprehensive Organic Transformation” published by Wily-CVH and edited by R. C. Larock.

The starting materials used and the intermediates prepared using the methods set forth in Schemes 1 and 2 above may be isolated and purified if desired using conventional techniques, including but not limited to filtration, distillation, crystallization, chromatography and alike. Such materials can be characterized using conventional means, including physical constants and spectral data.

### EXAMPLES

### General Methods

Solvents, reagents, and intermediates that are commercially available were used as received. Reagents and intermediates that are not commercially available were prepared in the manner as described below. ¹H NMR spectra were obtained on a Varian AS-400 (400 MHz) and are reported as ppm down field from Me₄Si with number of protons, multiplicities, and coupling constants in Hz indicated parenthetically. Where LC/MS data are presented, analyses was performed using an Applied Biosystems API-100 mass spectrometer and Shimadzu SCL-10A LC column: Altech platinum C18, 3 micron, 33 mm×7 mm ID; gradient flow: 0 min—10% CH₃CN, 5 min—95% CH₃CN, 7 min—95% CH₃CN, 7.5 min—10% CH₃CN, 9 min—stop. The retention time and observed parent ion are given. MS data were obtained using Agilent Technologies LC/MSD SL or 1100 series LC/MSD mass spectrometer. Final compounds were purified by PrepLC using the column of Varian Pursuit XRs C18 10 μm 250×21.2 mm and an eluent mixture of mobile phase A and B. The mobile phase A is composed of 0.1% TFA in H₂O and the mobile phase B is composed of CH₃CN (95%)/H₂O (5%)/TFA (0.1%). The mixture of mobile phase A and B was eluted through the column at a flow rate of 20 mL/min at room temperature. The purity of all the final discrete compounds was checked by LCMS using a Higgins Haisil HL C18 5 μm 150×4.6 mm column and an eluent mixture of mobile phase A and B, wherein mobile phase A is composed of 0.1% TFA in H₂O and the mobile phase B is composed of CH₃CN (95%)/H₂O (5%)/TFA (0.1%). The column was eluted at a flow rate of 3 mL/min at a temperature of 60° C. Intermediate compounds were characterized by LCMS using a Higgins Haisil HL C18 5 μm 50×4.6 mm column and an eluent mixture of mobile phase A and B, wherein mobile phase A is composed of 0.1% TFA in H₂O and the mobile phase B is composed of CH₃CN (95%)/H₂O (5%)/TFA (0.1%). The column was eluted at a flow rate of 3 mL/min at a column temperature of 60° C.

### Post Purification

Each final compound made in the Examples below was lyophilized after final purification as follows:

The compound was dissolved in 1 mL of acetonitrile and 1 mL of 1 N hydrochloric acid standard solution in water. The resulting solution was shaken for few minutes then transferred into a bar-coded 4 mL scintillation vial that was previously tared. The samples were lyophilized overnight then weighed and final yields were calculated.

### Example 1

### Preparation of Compounds 1-39

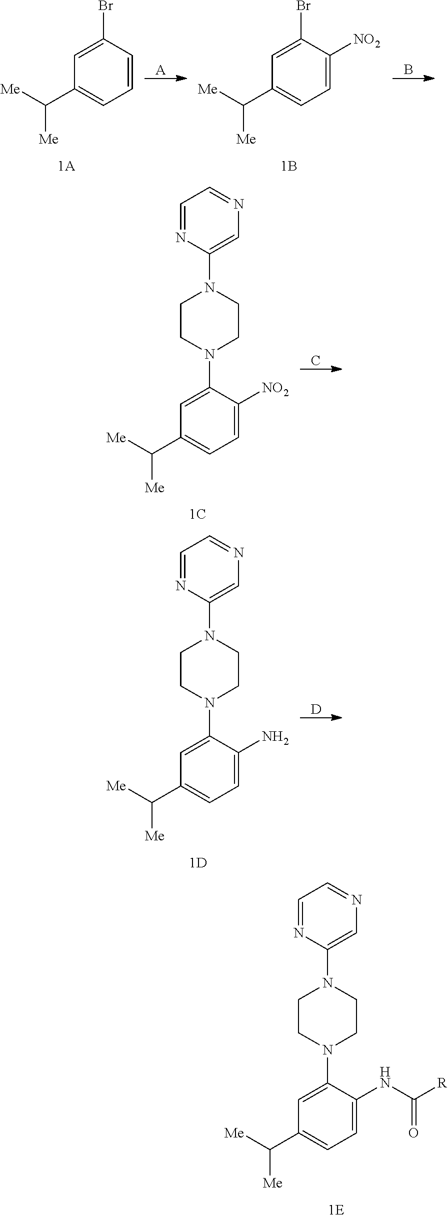

### Step A—Synthesis of Compound 1B

To the solution of compound 1A (4.28 g, 20 mmol) in toluene (150 mL) was slowly added m-chloroperoxybenzoic acid (25 g) in portions. The resulting reaction was then heated to reflux and allowed to stir at this temperature for about 15 hours. The reaction mixture was cooled to room temperature and filtered. The filtrate was diluted with ether, and washed with aqueous NaOH solution (10%) and brine, then dried over MgSO₄, filetered and concentrated in vacuo. The resulting residue was purified using flash column chromatography on silica gel (Hexane:DCM (4:1)) to provide compound 1B. ¹H NMR (400 MHz, CDCl₃) δ 7.82 (d, J=8.8 Hz, 1H), 7.58 (d, J=1.6 Hz, 1H), 7.29 (dd, J=8.4, 2.0 Hz, 1H), 2.96 (septet, J=7.2 Hz, 1H), 1.27 (d, J=7.2 Hz, 6H).

### Step B—Synthesis of Compound 1C

To a solution of compound 1B (1.22 g, 5 mmol) in DMF (15 mL) was added 2-(piperazin-1-yl)pyrazine (1 g, 6 mmol) and DIEA (1.3 mL, 7.5 mmol). The reaction mixture was put in a microwave oven at 200° C. for 20 minutes. The reaction mixture was then removed from the microwave oven, cooled to room temperature and concentrated in vacuo. The residue obtained was purified using flash column chromatography on silica gel (Hexane:EtOAc (7:3)) to provide compound 1B.

### Step C—Synthesis of Compound ID

To a solution of compound 1C (4 mmol) in ethanol was added zinc powder (10 g, 160 mmol) and calcium chloride (0.45 g, 4 mmol). The resulting reaction was then heated to reflux and allowed to stir at this temperature for 4 hours. The reaction mixture was cooled to room temperature and filtered through celite and the filtrate was concentrated in vacua to provide compound 1D, which was used without further purification.

### Step D—Synthesis of Compound of Formula 1E

To the solution of a representative carboxylic acid (0.02 mmol) in DMF was added HATU (7.6 mg, 0.02 mmol), followed by compound in (6 mg, 0.02 mmol) and DIEA (17 μL, 0.1 mmol). The resulting reaction was then heated to 80° C. and allowed to stir at this temperature for about 15 hours. The reaction mixture was then concentrated in vacuo and the residue obtained was purified using reverse phase HPLC to provide Compound 1E.

Using the above method and substituting the appropriate in Step D, the following compounds of the present invention were prepared:

| | | | Retention |
|---|---|---|---|
| Compound | | MS | Time |
| No. | Structure | (M⁺ + H) | (min) |
| 1 | 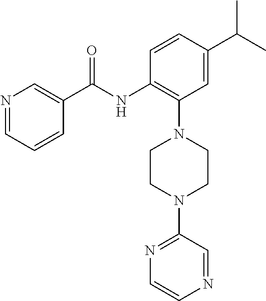 [SMILES: CC(C)c1ccc(NC(=O)c2cccnc2)c(N2CCN(c3cnccn3)CC2)c1] | 403.17 | 4.10 |
| 2 | 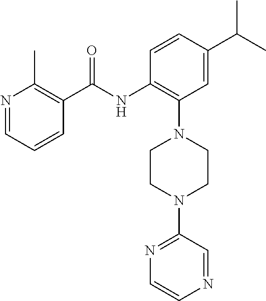 [SMILES: Cc1ncccc1C(=O)Nc1ccc(C(C)C)cc1N1CCN(c2cnccn2)CC1] | 417.25 | 3.84 |
| 3 | 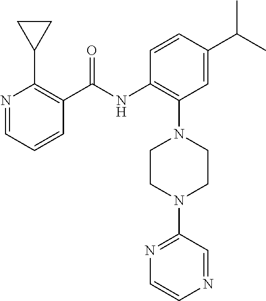 [SMILES: CC(C)c1ccc(NC(=O)c2cccnc2C2CC2)c(N2CCN(c3cnccn3)CC2)c1] | 443.25 | 4.27 |
| 4 | 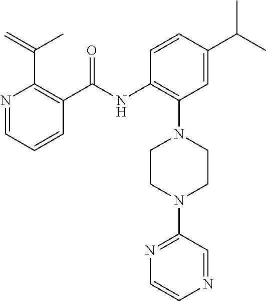 [SMILES: C=C(C)c1ncccc1C(=O)Nc1ccc(C(C)C)cc1N1CCN(c2cnccn2)CC1] | 443.25 | 3.85 |
| 5 | 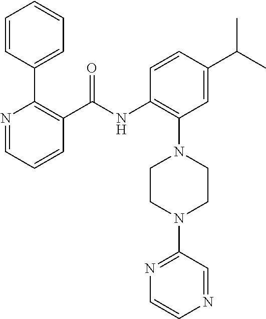 [SMILES: CC(C)c1ccc(NC(=O)c2cccnc2-c2ccccc2)c(N2CCN(c3cnccn3)CC2)c1] | 479.24 | 4.65 |
| 6 | 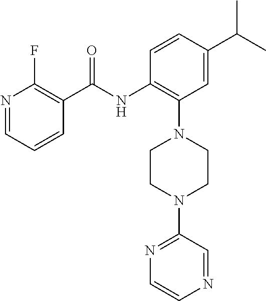 [SMILES: CC(C)c1ccc(NC(=O)c2cccnc2F)c(N2CCN(c3cnccn3)CC2)c1] | 421.66 | 4.86 |
| 7 | 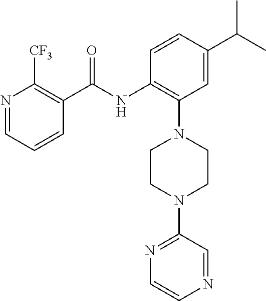 [SMILES: CC(C)c1ccc(NC(=O)c2cccnc2C(F)(F)F)c(N2CCN(c3cnccn3)CC2)c1] | 471.21 | 4.73 |
| 8 | 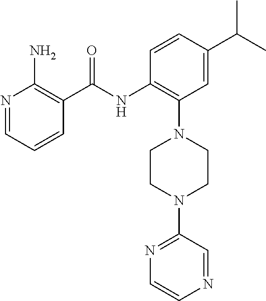 [SMILES: CC(C)c1ccc(NC(=O)c2cccnc2N)c(N2CCN(c3cnccn3)CC2)c1] | 418.25 | 3.72 |
| 9 | 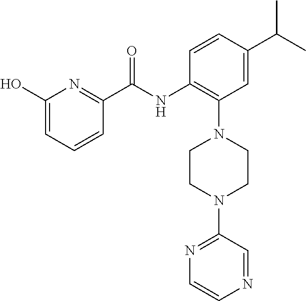 [SMILES: CC(C)c1ccc(NC(=O)c2cccc(O)n2)c(N2CCN(c3cnccn3)CC2)c1] | 419.18 | 4.28 |
| 10 | 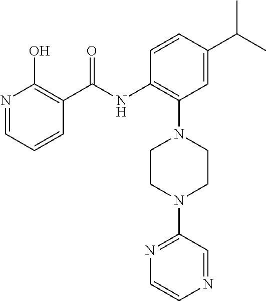 [SMILES: CC(C)c1ccc(NC(=O)c2cccnc2O)c(N2CCN(c3cnccn3)CC2)c1] | 419.18 | 4.20 |
| 11 | 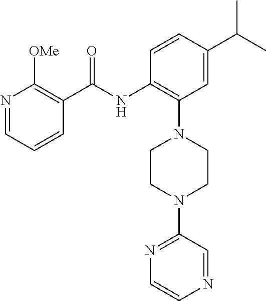 [SMILES: COc1ncccc1C(=O)Nc1ccc(C(C)C)cc1N1CCN(c2cnccn2)CC1] | 433.23 | 5.16 |
| 12 | 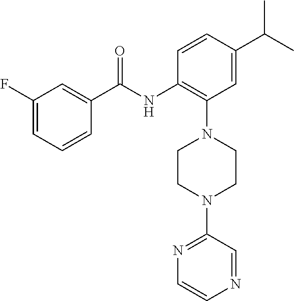 [SMILES: CC(C)c1ccc(NC(=O)c2cccc(F)c2)c(N2CCN(c3cnccn3)CC2)c1] | 420.23 | 5.21 |
| 13 | 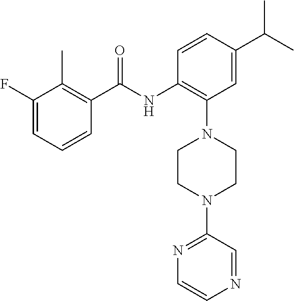 [SMILES: Cc1c(F)cccc1C(=O)Nc1ccc(C(C)C)cc1N1CCN(c2cnccn2)CC1] | 434.70 | 5.19 |
| 14 | 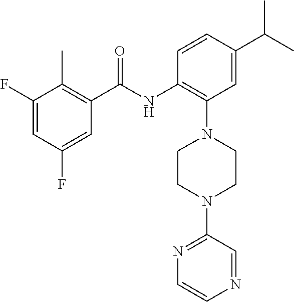 [SMILES: Cc1c(F)cc(F)cc1C(=O)Nc1ccc(C(C)C)cc1N1CCN(c2cnccn2)CC1] | 452.26 | 5.36 |
| 15 | 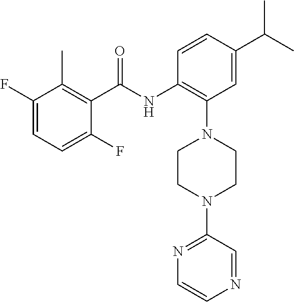 [SMILES: Cc1c(F)ccc(F)c1C(=O)Nc1ccc(C(C)C)cc1N1CCN(c2cnccn2)CC1] | 452.29 | 5.14 |
| 16 | 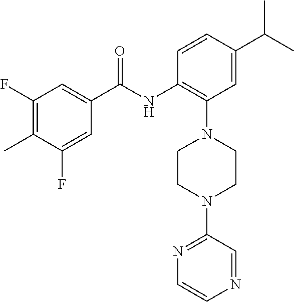 [SMILES: Cc1c(F)cc(C(=O)Nc2ccc(C(C)C)cc2N2CCN(c3cnccn3)CC2)cc1F] | 452.24 | 5.53 |
| 17 | 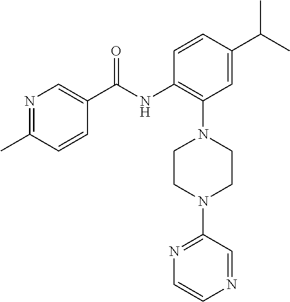 [SMILES: Cc1ccc(C(=O)Nc2ccc(C(C)C)cc2N2CCN(c3cnccn3)CC2)cn1] | 417.24 | 3.93 |
| 18 | 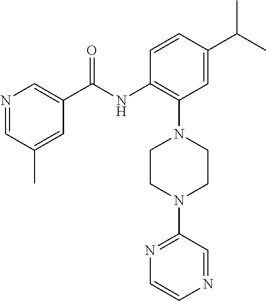 [SMILES: Cc1cncc(C(=O)Nc2ccc(C(C)C)cc2N2CCN(c3cnccn3)CC2)c1] | 417.24 | 4.12 |
| 19 | 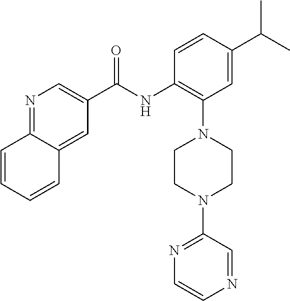 [SMILES: CC(C)c1ccc(NC(=O)c2cnc3ccccc3c2)c(N2CCN(c3cnccn3)CC2)c1] | 453.22 | 4.70 |
| 20 | 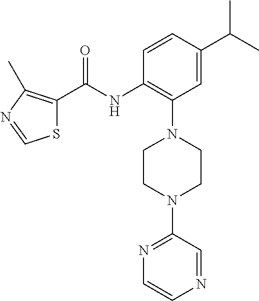 [SMILES: Cc1ncsc1C(=O)Nc1ccc(C(C)C)cc1N1CCN(c2cnccn2)CC1] | 423.63 | 4.77 |
| 21 | 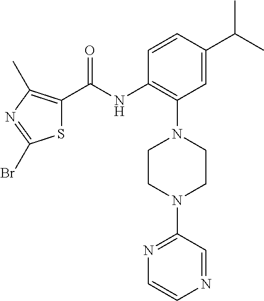 [SMILES: Cc1nc(Br)sc1C(=O)Nc1ccc(C(C)C)cc1N1CCN(c2cnccn2)CC1] | 501.10 | 5.45 |
| 22 | 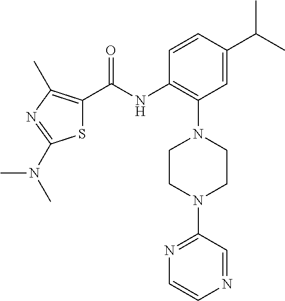 [SMILES: Cc1nc(N(C)C)sc1C(=O)Nc1ccc(C(C)C)cc1N1CCN(c2cnccn2)CC1] | 466.21 | 4.41 |
| 23 | 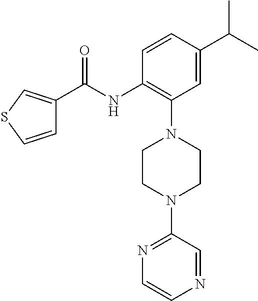 [SMILES: CC(C)c1ccc(NC(=O)c2ccsc2)c(N2CCN(c3cnccn3)CC2)c1] | 408.18 | 4.90 |
| 24 | 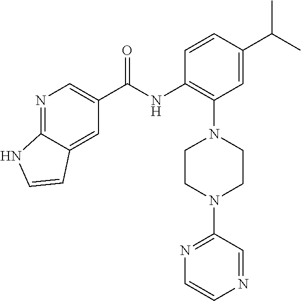 [SMILES: CC(C)c1ccc(NC(=O)c2cnc3[nH]ccc3c2)c(N2CCN(c3cnccn3)CC2)c1] | 442.22 | 4.45 |
| 25 | 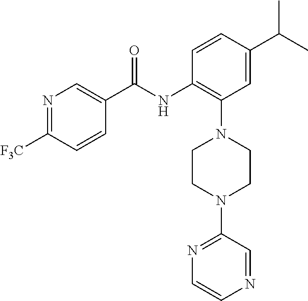 [SMILES: CC(C)c1ccc(NC(=O)c2ccc(C(F)(F)F)nc2)c(N2CCN(c3cnccn3)CC2)c1] | 471.22 | 5.17 |
| 26 | 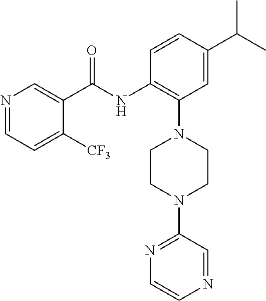 [SMILES: CC(C)c1ccc(NC(=O)c2cnccc2C(F)(F)F)c(N2CCN(c3cnccn3)CC2)c1] | 471.18 | 4.93 |
| 27 | 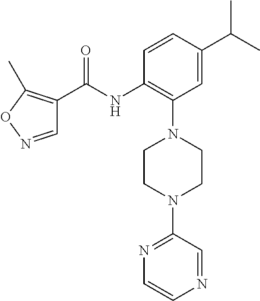 [SMILES: Cc1oncc1C(=O)Nc1ccc(C(C)C)cc1N1CCN(c2cnccn2)CC1] | 407.66 | 5.08 |
| 28 | 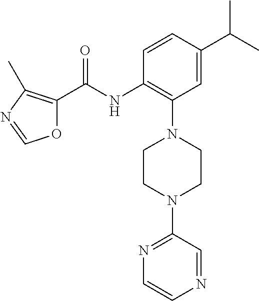 [SMILES: Cc1ncoc1C(=O)Nc1ccc(C(C)C)cc1N1CCN(c2cnccn2)CC1] | 407.68 | 4.77 |
| 29 | 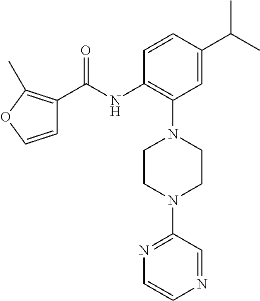 [SMILES: Cc1occc1C(=O)Nc1ccc(C(C)C)cc1N1CCN(c2cnccn2)CC1] | 406.66 | 4.88 |
| 30 | 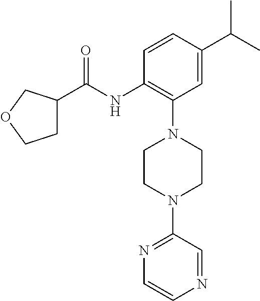 [SMILES: CC(C)c1ccc(NC(=O)C2CCOC2)c(N2CCN(c3cnccn3)CC2)c1] | 396.69 | 4.17 |
| 31 | 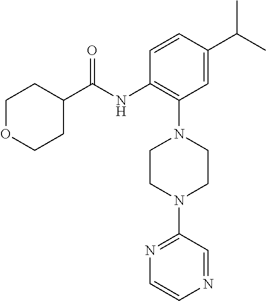 [SMILES: CC(C)c1ccc(NC(=O)C2CCOCC2)c(N2CCN(c3cnccn3)CC2)c1] | 410.69 | 4.24 |
| 32 | 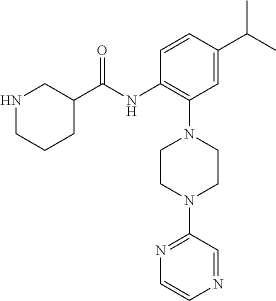 [SMILES: CC(C)c1ccc(NC(=O)C2CCCNC2)c(N2CCN(c3cnccn3)CC2)c1] | 409.71 | 3.51 |
| 33 | 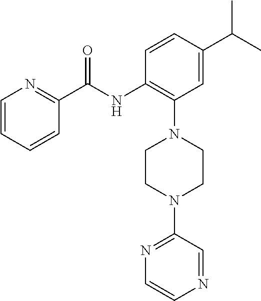 [SMILES: CC(C)c1ccc(NC(=O)c2ccccn2)c(N2CCN(c3cnccn3)CC2)c1] | 403.24 | 5.23 |
| 34 | 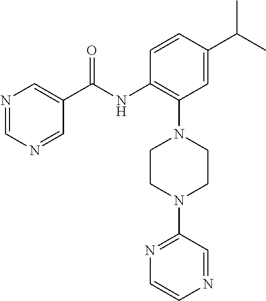 [SMILES: CC(C)c1ccc(NC(=O)c2cncnc2)c(N2CCN(c3cnccn3)CC2)c1] | 404.22 | 4.30 |
| 35 | 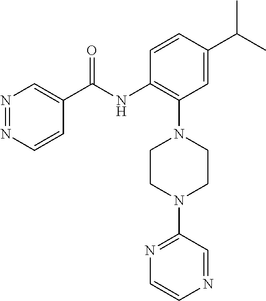 [SMILES: CC(C)c1ccc(NC(=O)c2ccnnc2)c(N2CCN(c3cnccn3)CC2)c1] | 404.23 | 4.24 |
| 36 | 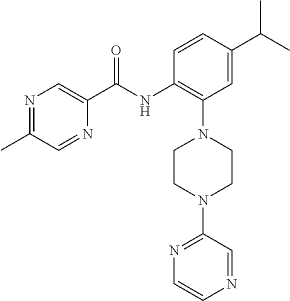 [SMILES: Cc1cnc(C(=O)Nc2ccc(C(C)C)cc2N2CCN(c3cnccn3)CC2)cn1] | 418.23 | 5.15 |
| 37 | 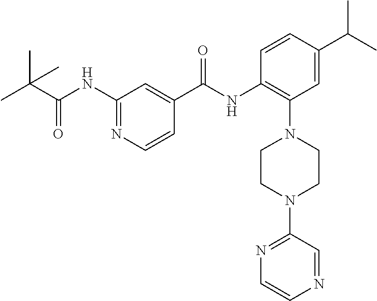 [SMILES: CC(C)c1ccc(NC(=O)c2ccnc(NC(=O)C(C)(C)C)c2)c(N2CCN(c3cnccn3)CC2)c1] | 502.25 | 5.35 |
| 38 | 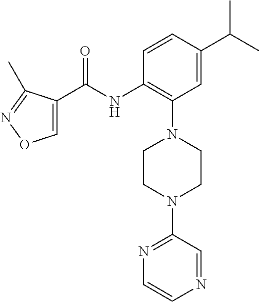 [SMILES: Cc1nocc1C(=O)Nc1ccc(C(C)C)cc1N1CCN(c2cnccn2)CC1] | 407.65 | 4.52 |
| 39 | 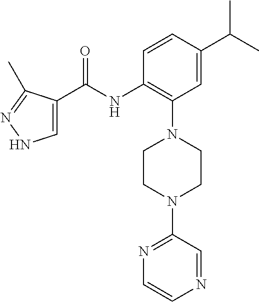 [SMILES: Cc1n[nH]cc1C(=O)Nc1ccc(C(C)C)cc1N1CCN(c2cnccn2)CC1] | 406.70 | 4.17 |

### Example 2

### Preparation of Compounds 40-57

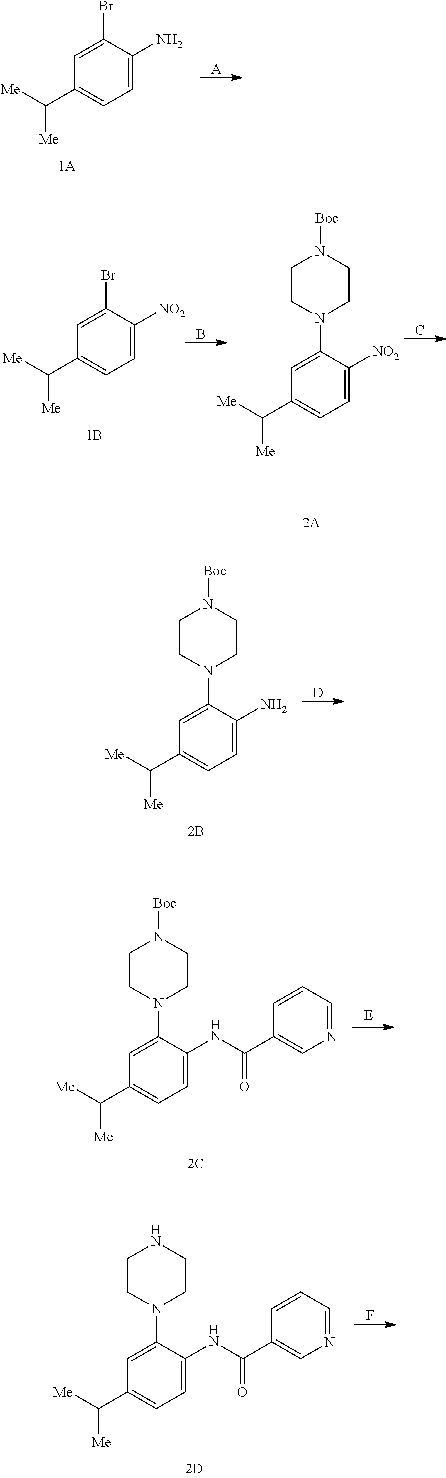

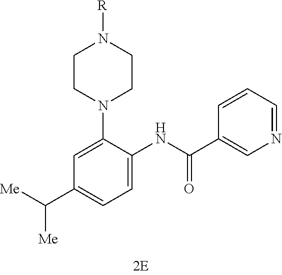

### Step A—Synthesis of Compound 1B

Using the method described in Example 1, Step A, compound 1A was converted to compound 1B.

### Step B—Synthesis of Compound 2A

To a solution of compound 1B (1.22 g, 5 mmol) in DMF (15 mL) was added tert-butyl piperazine-1-carboxylate (1.2 g, 6 mmol), and DIEA (1.3 mL, 7.5 mmol). The reaction mixture was put in a Biotage microwave oven and heated at 200° C. for 20 minutes. The reaction mixture was then removed from the microwave oven, cooled to room temperature and concentrated in vacuo. The residue obtained was purified using flash column chromatography on silica gel (Hexane:EtOAc (7:3)) to provide compound 2A. ¹H NMR (400 MHz, CDCl₃) δ 7.78 (d, J=8.8 Hz, 1H), 6.96-6.91 (m, 2H), 3.64-3.54 (m, 4H), 3.09-2.98 (m, 4H), 2.92 (septet, J=6.8 Hz, 1H), 1.48 (s, 9H), 1.25 (d, J =6.8 Hz, 6H).

### Step C—Synthesis of Compound 2B

To a solution of compound 2A (1 g) in methanol (30 mL) was added palladium on carbon (10%, 0.5 g). The reaction was stirred under an atmosphere of hydrogen at room temperature for about 15 hours. The reaction mixture was filtered through celite and the filtrate was concentrated in vacuo to provide compound 2B (711 mg), which was used without further purification.

### Step D—Synthesis of Compound 2C

To a solution of nicotinic acid (150 mg, 1.2 mmol) in DMF (5 mL) was added HATU (456 mg, 1.2 mmol), followed compound 2B (320 mg, 1 mmol) and DIEA (260 μL, 1.5 mmol). The reaction mixture was heated to 80° C. and allowed to stir at this temperature for about 15 hours. The reaction mixture was cooled to room temperature and concentrated in vacuo and the resulting residue was purified using flash column chromatography on silica gel using EtOAc as the eluent to provide 160 mg (80% yield) of compound 2C.

### Step E—Synthesis of Compound 2D

Compound 2C (160 mg) was treated with TFA (neat) (2 mL). The mixture was stirred at room temperature for 30 minutes, then concentrated in vacuo to provide compound 2D, which was used without further purification.

### Step F—Synthesis of a Compound of Formula 2E

To a solution of compound 2D (6.5 mg, 0.02 mmol) in DMF was added a representative electrophile (0.02 mmol) and DIEA (17 μL, 0.1 mmol). The reaction mixture was stirred in microwave at 200° C. for 20 minutes. The reaction mixture was cooled to room temperature and concentrated in vacuo and the resulting residue was purified using reverse phase HPLC to provide Compound 2E.

Using the above method and substituting the appropriate starting materials and reagents, the following compounds of the present invention were prepared:

| | | | Reten- |
|---|---|---|---|
| Com- | | | tion |
| pound | | MS m/z | Time |
| No. | Structure | (M⁺ + H) | (min) |
| 40 | 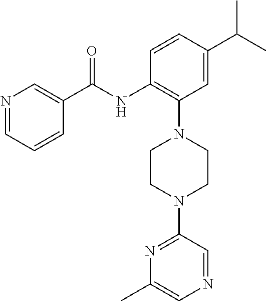 [SMILES: Cc1cncc(N2CCN(c3cc(C(C)C)ccc3NC(=O)c3cccnc3)CC2)n1] | 417.70 | 4.14 |
| 41 | 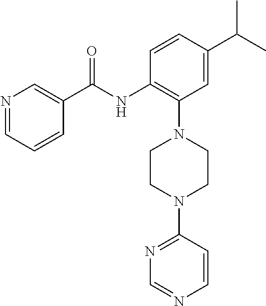 [SMILES: CC(C)c1ccc(NC(=O)c2cccnc2)c(N2CCN(c3ccncn3)CC2)c1] | 403.69 | 3.12 |
| 42 | 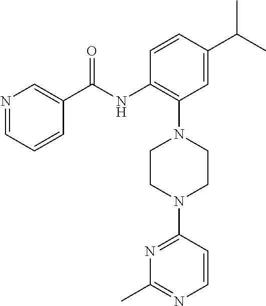 [SMILES: Cc1nccc(N2CCN(c3cc(C(C)C)ccc3NC(=O)c3cccnc3)CC2)n1] | 417.16 | 3.28 |
| 43 | 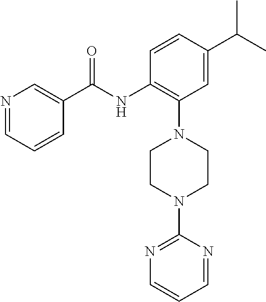 [SMILES: CC(C)c1ccc(NC(=O)c2cccnc2)c(N2CCN(c3ncccn3)CC2)c1] | 403.66 | 3.92 |
| 44 | 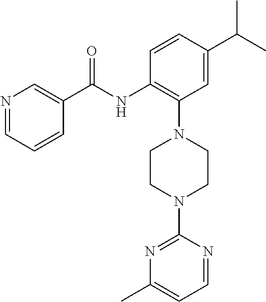 [SMILES: Cc1ccnc(N2CCN(c3cc(C(C)C)ccc3NC(=O)c3cccnc3)CC2)n1] | 417.26 | 3.90 |
| 45 | 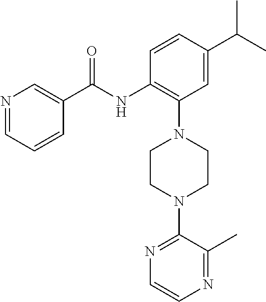 [SMILES: Cc1nccnc1N1CCN(c2cc(C(C)C)ccc2NC(=O)c2cccnc2)CC1] | 417.25 | 4.19 |
| 46 | 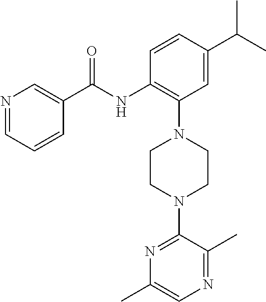 [SMILES: Cc1cnc(C)c(N2CCN(c3cc(C(C)C)ccc3NC(=O)c3cccnc3)CC2)n1] | 431.26 | 4.21 |
| 47 | 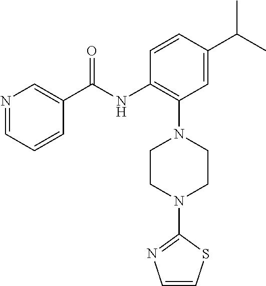 [SMILES: CC(C)c1ccc(NC(=O)c2cccnc2)c(N2CCN(c3nccs3)CC2)c1] | 408.65 | 3.29 |
| 48 | 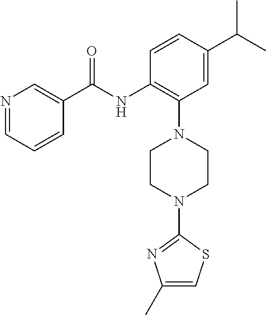 [SMILES: Cc1csc(N2CCN(c3cc(C(C)C)ccc3NC(=O)c3cccnc3)CC2)n1] | 422.16 | 3.42 |
| 49 | 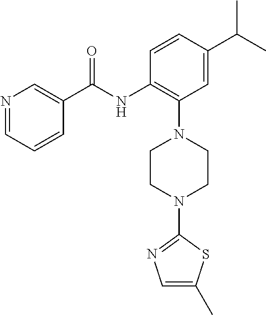 [SMILES: Cc1cnc(N2CCN(c3cc(C(C)C)ccc3NC(=O)c3cccnc3)CC2)s1] | 422.22 | 3.46 |
| 50 | 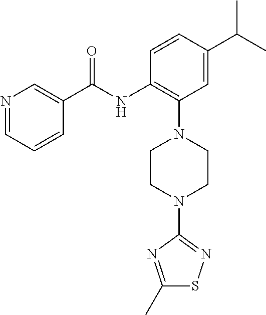 [SMILES: Cc1nc(N2CCN(c3cc(C(C)C)ccc3NC(=O)c3cccnc3)CC2)ns1] | 443.15 | 4.44 |
| 51 | 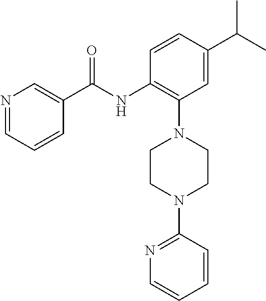 [SMILES: CC(C)c1ccc(NC(=O)c2cccnc2)c(N2CCN(c3ccccn3)CC2)c1] | 402.65 | 3.15 |
| 52 | 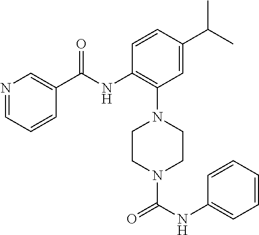 [SMILES: CC(C)c1ccc(NC(=O)c2cccnc2)c(N2CCN(C(=O)Nc3ccccc3)CC2)c1] | 444.68 | 4.07 |
| 53 | 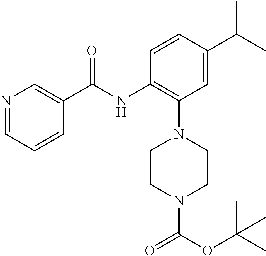 [SMILES: CC(C)c1ccc(NC(=O)c2cccnc2)c(N2CCN(C(=O)OC(C)(C)C)CC2)c1] | 425.25 | 4.73 |
| 54 | 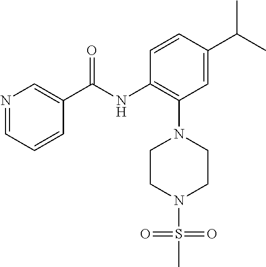 [SMILES: CC(C)c1ccc(NC(=O)c2cccnc2)c(N2CCN(S(C)(=O)=O)CC2)c1] | 403.63 | 3.64 |
| 55 | 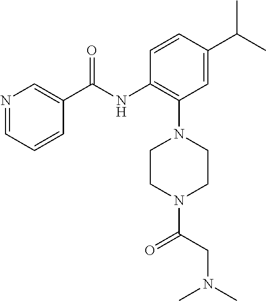 [SMILES: CC(C)c1ccc(NC(=O)c2cccnc2)c(N2CCN(C(=O)CN(C)C)CC2)c1] | 410.70 | 2.90 |
| 56 | 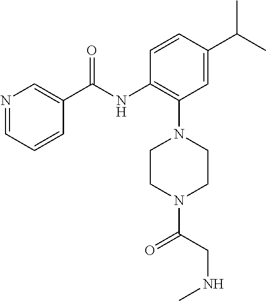 [SMILES: CNCC(=O)N1CCN(c2cc(C(C)C)ccc2NC(=O)c2cccnc2)CC1] | 396.69 | 2.81 |

### Example 3

### Preparation of Compounds 57-60

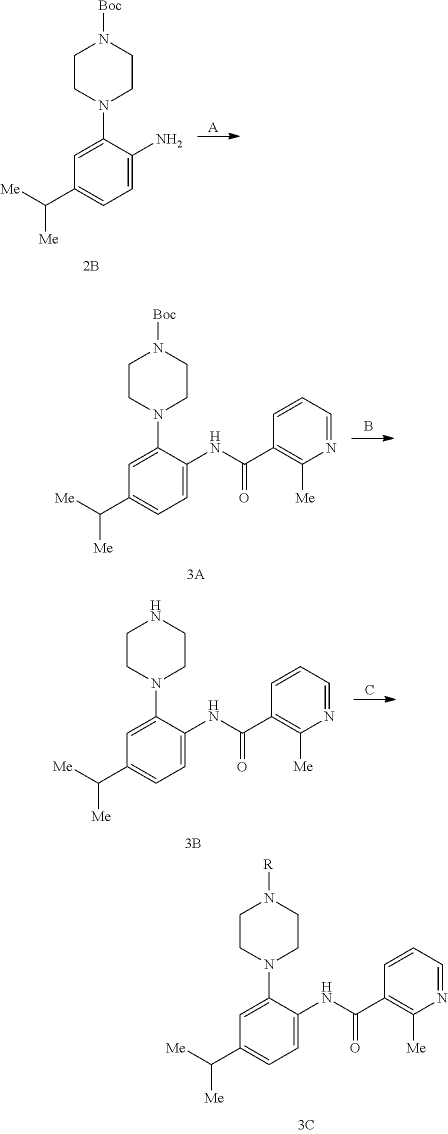

### Step A—Synthesis of Compound 3A

Using the method described in Example 2, Step D and substituting 2-methylnicotinic acid for nicotinic acid, compound 3A was prepared and purified using flash column chromatography on silica gel using a mixture of EtOAc:Et₃N:MeOH (96:2:2) as the eluent.

### Step B—Synthesis of Compound 3B

Compound 3B was prepared using the method described in Example 2, Step E and substituting compound 3A for compound 2C.

### Step C—Synthesis of Compound of Formula 3C

Using the method described in Example 2, Step E and substituting compound 3B for compound 2D, the compounds of formula 3C were prepared and subsequently purified using reverse phase HPLC.

Using the above method and substituting the appropriate starting materials and reagents, the following compounds of the present invention were prepared:

| | | | Reten- |
|---|---|---|---|
| Com- | | | tion |
| pound | | MS | Time |
| No. | Structure | (M⁺ + H) | (min) |
| 57 | 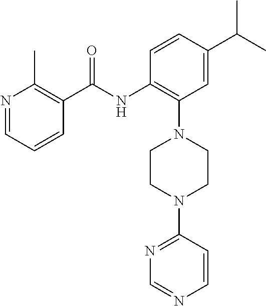 [SMILES: Cc1ncccc1C(=O)Nc1ccc(C(C)C)cc1N1CCN(c2ccncn2)CC1] | 417.25 | 2.86 |
| 58 | 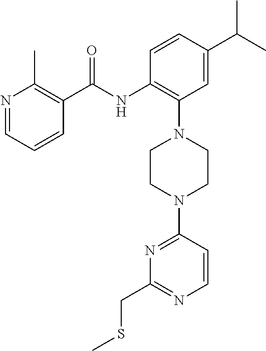 [SMILES: CSCc1nccc(N2CCN(c3cc(C(C)C)ccc3NC(=O)c3cccnc3C)CC2)n1] | 477.22 | 3.26 |
| 59 | 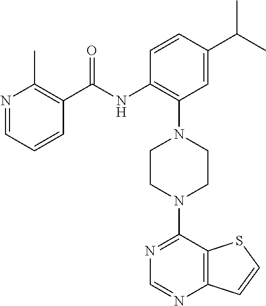 [SMILES: Cc1ncccc1C(=O)Nc1ccc(C(C)C)cc1N1CCN(c2ncnc3ccsc23)CC1] | 473.21 | 3.09 |
| 60 | 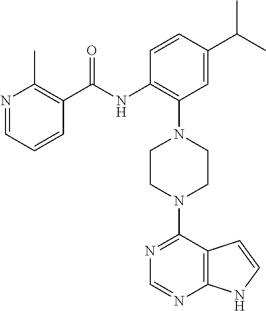 [SMILES: Cc1ncccc1C(=O)Nc1ccc(C(C)C)cc1N1CCN(c2ncnc3[nH]ccc23)CC1] | 456.22 | 2.99 |

### Example 4

### Preparation of Compounds 61 and 62

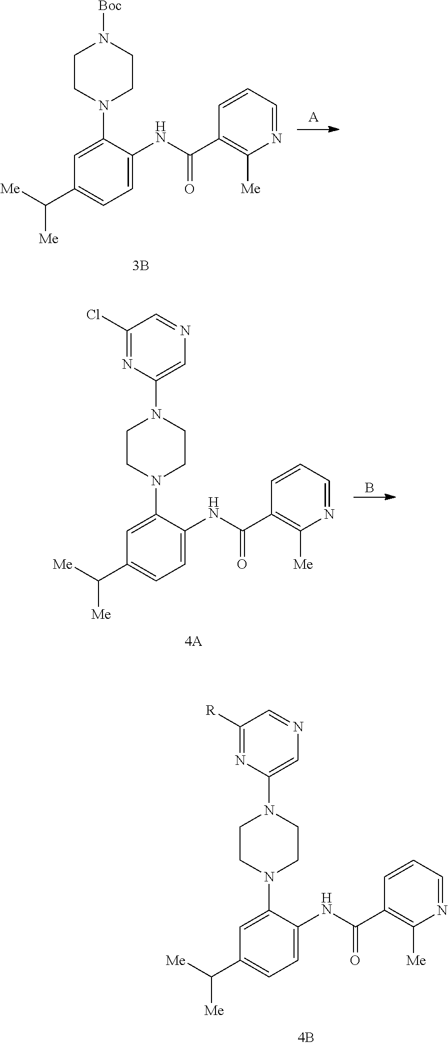

### Step A—Synthesis of Compound 4A

To a solution of compound 3B (135 mg, 0.4 mmol) in DMF (2 mL) was added 2,6-dichloropyrazine (60 mg, 0.4 mmol) and DIEA (175 μL, 1 mmol). The reaction was stirred in microwave at 200° C. for 20 minutes. The mixture was cooled to room temperature and concentrated. The residue was purified using flash column chromatography on silica gel using a mixture of EtOAc:Et₃N:MeOH (96:2:2) as the eluent to provide compound 4A in the amount of 100 mg.

### Step B—Synthesis of a Compound of Formula 4B

A mixture of compound 4A (13.5 mg, 0.03 mmol), boronic acid (0.05 mmol), Pd₂(DBA)₃ (2.75 mg, 0.003 mmol), S-Phos (2.5 mg, 0.006 mmol) and K₃PO₄ (12.7 mg, 0.06 mmol) in toluene (0.5 mL) was put under argon atmosphere, then heated to 100° C. and allowed to stir at this temperature for about 15 hours. The reaction mixture was cooled to room temperature, filtered and concentrated in vacua to provide a compound of formula 4B, which was subsequently purified using reverse phase HPLC.

Using the above method and substituting the appropriate starting materials and reagents, the following compounds of the present invention were prepared:

| | | | Reten- |
|---|---|---|---|
| Com- | | | tion |
| pound | | MS m/z | Time |
| No. | Structure | (M⁺ + H) | (min) |
| 61 | 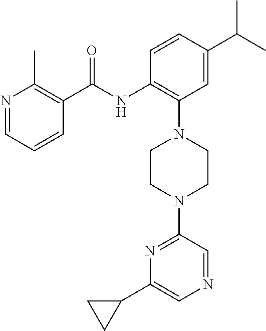 [SMILES: Cc1ncccc1C(=O)Nc1ccc(C(C)C)cc1N1CCN(c2cncc(C3CC3)n2)CC1] | 457.24 | 4.39 |
| 62 | 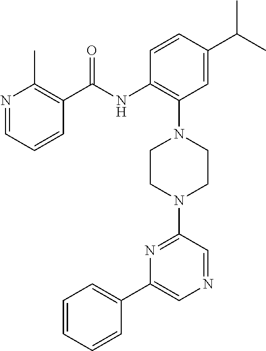 [SMILES: Cc1ncccc1C(=O)Nc1ccc(C(C)C)cc1N1CCN(c2cncc(-c3ccccc3)n2)CC1] | 493.27 | 4.77 |

### Example 5

### Preparation of Compound 63

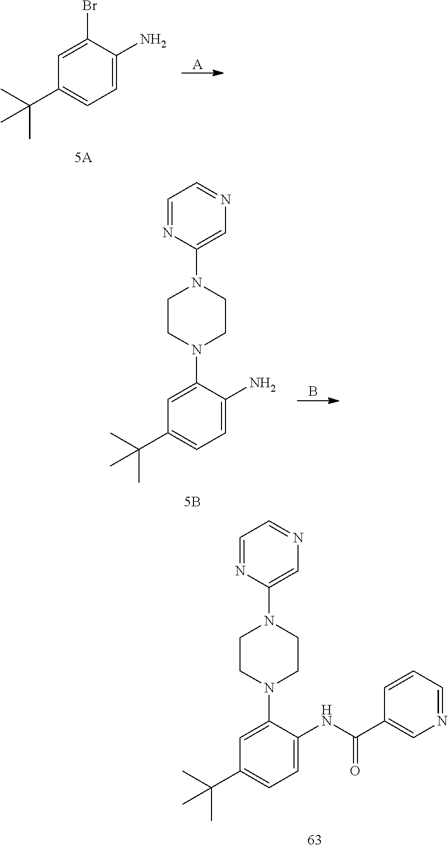

### Step A—Synthesis of Compound 5B

A mixture of compound 5A (230 mg, 1 mmol), 2-(piperazin-1-yl)pyrazine (164 mg, 1 mmol), CuI (19 mg, 0.1 mmol), biphenyl-2-ol (34 mg, 0.2 mmol) and K₃PO₄ (424 mg, 2 mmol) in toluene (0.5 mL) was put under argon atmosphere, then heated to 110° C. and allowed to stir at this temperature for about 15 hours. The reaction mixture was cooled to room temperature, filtered and concentrated in vacuo and the resulting residue was purified using reverse phase HPLC to provide Compound 5B (50 mg, 16% yield).

### Step B—Synthesis of Compound 63

Using the method described in Example 2, Step D and substituting compound 5B for compound 2B, compound 63 was prepared and purified using reverse phase HPLC. (1 mg, 5% yield).

| Compound | MS m/z | Retention |
|---|---|---|
| No. | (M⁺ + H) | Time (min) |
| 63 | 417.22 | 4.31 |

### Example 6

### Preparation of Compounds 64 and 65

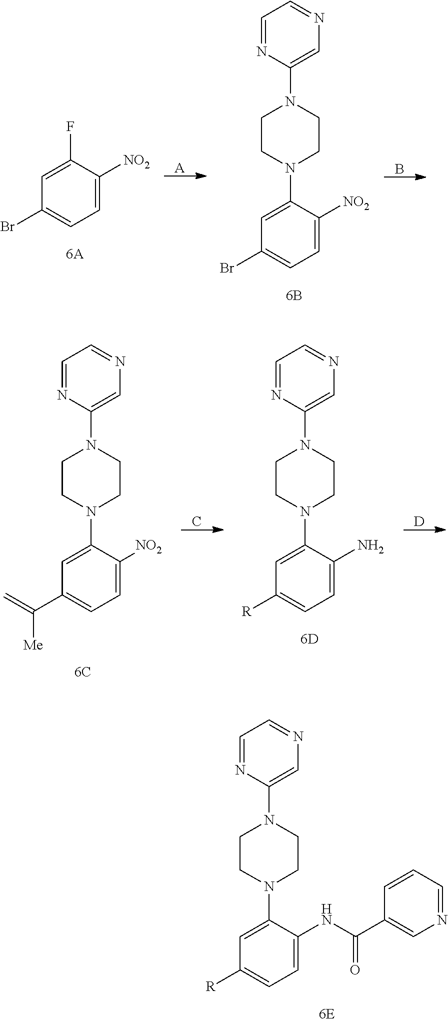

### Step A—Synthesis of Compound 6B

A mixture of compound 6A (440 mg, 2 mmol), 2-(piperazin-1-yl)pyrazine (410 mg, 2.5 mmol) and DIEA (523 μL, 3 mmol) in DMF was stirred in a microwave at 120° C. for 20 minutes. The reaction mixture was cooled to room temperature and concentrated in vacuo and the resulting residue was purified using flash column chromatography on silica gel (Hexane:EtOAc (2:1)) to provide compound 6B as a yellow solid (550 mg, 75% yield).

### Step B—Synthesis of Compound 6C

A mixture of compound 6B (546 mg, 1.5 mmol), Pd₂(DBA)₃ (46 mg, 0.05 mmol), RuPhos (70 mg, 0.15 mmol) and isopropenylzinc chloride (2 mmol, 4 mL of 0.5 M in THF solution prepared from isopropenylmagnesium bromide and zinc chloride in THF solution) was placed in a sealed Schlenk tube under Ar. The reaction mixture was heated to 80° C. and allowed to stir at this temperature for about 15 hours. The reaction mixture was cooled to room temperature and concentrated in vacuo and the resulting residue was purified using flash column chromatography on silica gel (Hexane:EtOAc (2:1)) to provide mixture of compound 6C and compound 21. (R=isopropenyl). (30 mg, 6% yield by NMR analysis).

### Step C—Synthesis of Compounds of Formula 6D

Using the method described in Example 1, Step C and substituting a mixture of compound 6C and compound 21 (R=isopropenyl) for compound 1C, compound 6D (R=isopropenyl, Br) was prepared.

### Step D—Synthesis of Compounds of Formula 6E

Using the method described in Example 1, Step D and substituting compound 6D for compound 1D, compound 6E (R=isopropenyl, Br) was prepared and purified using reverse phase HPLC. (1.78 mg of 64 and 11 mg of 65).

Using the above method and substituting the appropriate starting materials and reagents, the following compounds of the present invention were prepared:

| | | | Reten- |
|---|---|---|---|
| Com- | | | tion |
| pound | | MS m/z | Time |
| No. | Structure | (M⁺ + H) | (min) |
| 64 | 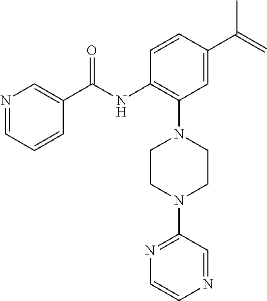 [SMILES: C=C(C)c1ccc(NC(=O)c2cccnc2)c(N2CCN(c3cnccn3)CC2)c1] | 401.18 | 4.03 |
| 65 | 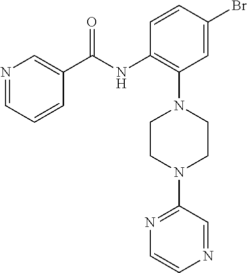 [SMILES: O=C(Nc1ccc(Br)cc1N1CCN(c2cnccn2)CC1)c1cccnc1] | 439.08 | 3.91 |

### Example 7

### Preparation of Compounds 66 and 67

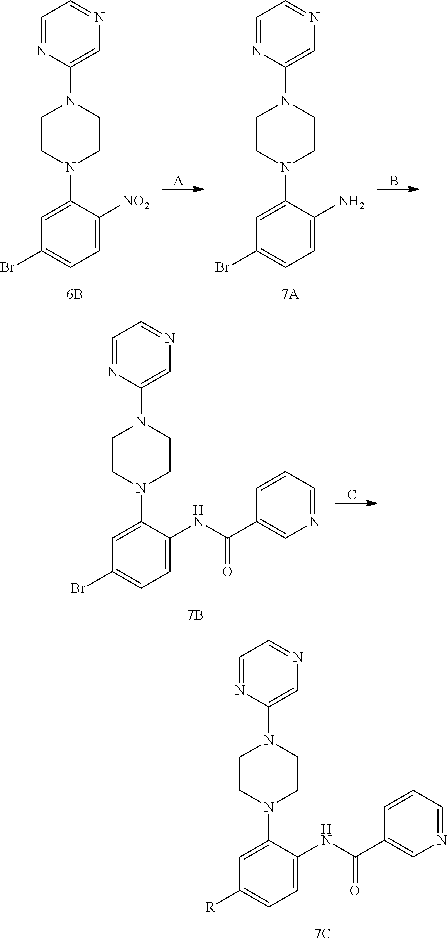

### Step A—Synthesis of Compound 7A

Compound 7A was prepared using the method described in Example 2, Step C and substituting compound 6B (1 g, 2.8 mmole) for compound 2A. (7A, 450 mg, 50% yield).

### Step B—Synthesis of Compound 7B

Using the method described in Example 1, Step D and substituting compound 7A (450 mg, 1.35 mmole) for compound 1D, compound 7B was prepared and purified using reverse phase HPLC. (7B, 60 mg, 10% yield).

### Step D—Synthesis of Compound 7C

A mixture of compound 7B (28 mg, 0.05 mmol), Pd₂(DBA)₃ (1.8 mg, 0.002 mmol), X-Phos (2.4 mg, 0.005 mmol), RB(OH)₂ (0.1 mmole, R=isopropyl (66); Ph (67)) and K₃PO₄ (21.2 mg, 0.1 mmol) in toluene was placed in a sealed Schlenk tube under Ar. The reaction was heated to 100° C. and allowed to stir at this temperature for about 15 hours. The reaction mixture was cooled to mom temperature then filtered, and the filtrate was concentrated in vacuo. The resulting residue was purified using reverse phase HPLC to provide compound 7C (R=isopropyl (66); Ph (67)).

Using the above method and substituting the appropriate starting materials and reagents, the following compounds of the present invention were prepared:

| | | | Reten- |
|---|---|---|---|
| Com- | | | tion |
| pound | | MS m/z | Time |
| No. | Structure | (M⁺ + H) | (min) |
| 66 | 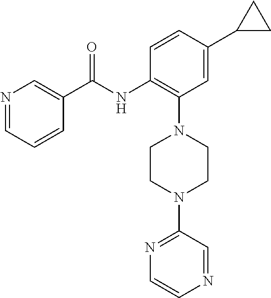 [SMILES: O=C(Nc1ccc(C2CC2)cc1N1CCN(c2cnccn2)CC1)c1cccnc1] | 401.26 | 3.77 |
| 67 | 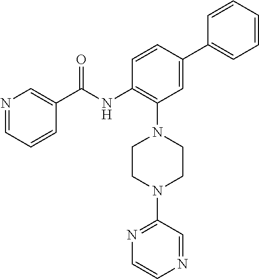 [SMILES: O=C(Nc1ccc(-c2ccccc2)cc1N1CCN(c2cnccn2)CC1)c1cccnc1] | 437.17 | 4.30 |

### Example 8

### Preparation of Compounds 68 and 69

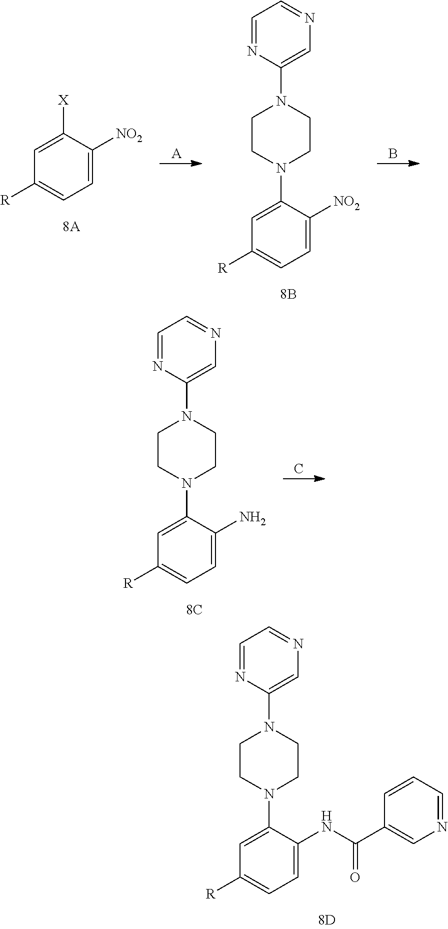

### Step A—Synthesis of Compound 8B

A mixture of compound 8A (where R═CF₃ and X═Cl for compound 68; R═CO₂Et and X═F for compound 69) (1 mmol), 2-(piperazin-1-yl)pyrazine (197 mg, 1.2 mmol) and DIEA (350 μL, 2 mmol) in DMF (2 mL) was stirred in microwave at 180° C. for 20 minutes. The mixture was cooled to room temperature and concentrated in vacuo to provide compound 8B in quantative yield, which was used without further purification.

### Step B—Synthesis of Compound 8C

Using the method described in Example 2, Step C and substituting compound 8B for compound 2A, compound 8C was prepared and purified using flash column chromatography on silica gel using an eluent mixture of Hexane:EtOAe (1:1). (where R═CF₃, 30% yield, and R═CO₂Et, 15% yield).

### Step C—Synthesis of Compound 8D

Using the method described in Example 1, Step D and substituting compound 8C for compound 1D, compound 8D was prepared and purified using reverse phase HPLC. (where R═CF₃, 2% yield, and R═CO₂Et, 5% yield)

Using the above method and substituting the appropriate starting materials and reagents, the following compounds of the present invention were prepared:

| | | | Reten- |
|---|---|---|---|
| Com- | | | tion |
| pound | | MS m/z | Time |
| No. | Structure | (M⁺ + H) | (min) |
| 68 | 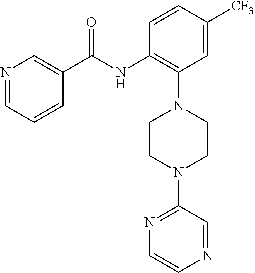 [SMILES: O=C(Nc1ccc(C(F)(F)F)cc1N1CCN(c2cnccn2)CC1)c1cccnc1] | 429.16 | 4.11 |
| 69 | 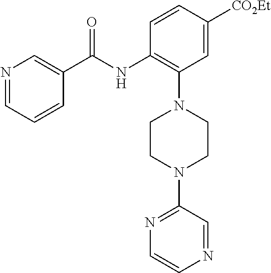 [SMILES: CCOC(=O)c1ccc(NC(=O)c2cccnc2)c(N2CCN(c3cnccn3)CC2)c1] | 433.27 | 3.78 |

### Example 9

### Preparation of Compound 70

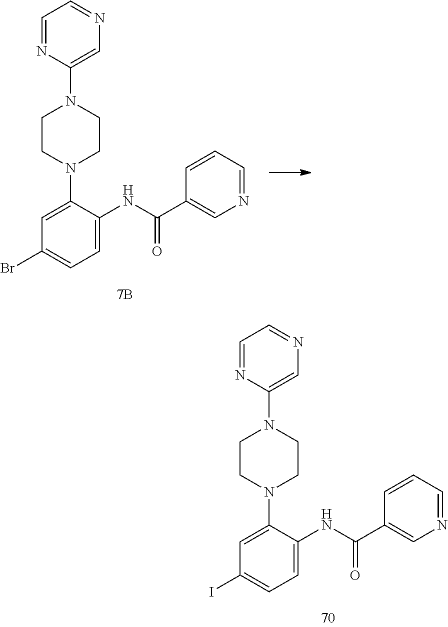 [SMILES: O=C(Nc1ccc(Br)cc1N1CCN(c2cnccn2)CC1)c1cccnc1 ; O=C(Nc1ccc(I)cc1N1CCN(c2cnccn2)CC1)c1cccnc1]

A mixture of compound 7B (4.38 mg, 0.01 mmol), CuI (0.19 mg, 0.001 mmol), N,N′-dimethylethylenediamine (0.22 μL, 0.002 mmol) and NaI (3 mg, 0.02 mmol) in dioxane was placed in a sealed Schlenk tube under Ar. The reaction was heated to 100° C. and allowed to stir at this temperature for about 15 hours. The reaction mixture was then cooled to room temperature and filtered, and the filtrate was concentrated in vacuo. The residue obtained was purified using reverse phase HPLC to provide compound 70. (0.6 mg, 10% yield).

| Compound | MS m/z | Retention |
|---|---|---|
| No. | (M + H) | Time (min) |
| 70 | 487.05 | 3.72 |

### Example 10

### HCV NS5B Polymerase Inhibition Assay

An in vitro transcribed heteropolymeric RNA known as D-RNA or DCoH has been shown to be an efficient template for HCV NS5B polymerase (S.-E. Behrens et al., EMBO J. 15:12-22 (1996); WO 96/37619). A chemically synthesized 75-mer version, designated DCoH75, whose sequence matches the 3′-end of D-RNA, and DCoH75ddC, where the 3′-terminal cytidine of DCoH75 is replaced by dideoxycytidine, were used for assaying the NS5B enzyme activity as described in Ferrari et al., 12^{th} International Symposium on HCV and Related Viruses, P-306 (2005). The sequence of the template RNA was: 5′-UGU GCC GGU CUU UCU GAA CGG GAU AUA AAC CUG GCC AGC UUC AUC GAA CAA GUU GCC GUG UCU AUG ACA UAG AUC-3′ (SEQ ID NO: 1). A soluble C-terminal 21-amino acid truncated NS5B enzyme form (NS5BΔCT21, from HCV-Con 1 isolate, genotype 1b, Genbank accession number AJ238799) was produced and purified from Escherichia coli as C-terminal polyhistidine-tagged fusion protein as described in Ferrari et al., J. Virol. 73:1649-1654 (1999). A typical assay contained 20 mM Hepes pH 7.3, 10 mM MgCl₂, 60 mM NaCl, 100 μg/ml BSA, 20 units/ml RNasin, 7.5 mM DTT, 0.1 μM ATP/GTP/UTP, 0.026 μM CTP, 0.25 mM GAU, 0.03 μM RNA template, 20 μCi/ml [³³P]-CTP, 2% DMSO, and 30 or 150 nM NS5B enzyme. Reactions were incubated at 22° C. for 2 hours, then stopped by adding 150 mM EDTA, washed in DE81 filter plate in 0.5M di-basic sodium phosphate buffer, pH 7.0, and counted using Packard TopCount after the addition of scintillation cocktail. Polynucleotide synthesis was monitored by the incorporation of radiolabeled CTP. The effect of the Compounds of Formula (I) on the polymerase activity was evaluated by adding various concentrations of a Compound of Formula (I), typically in 10 serial 2-fold dilutions, to the assay mixture. The starting concentrations ranged from 200 μM to 1 μM. An IC₅₀ value for the inhibitor, defined as the compound concentration that provides 50% inhibition of polymerase activity, was determined by fitting the cpm data to the Hill equation Y=100/(1+10̂((LogIC50−X)*HillSlope)), where X is the logarithm of compound concentration, and Y is the % inhibition. Ferrari et al., 12^{th} International Symposium on HCV and Related Viruses, P-306 (2005) described in detail this assay procedure. It should be noted that such an assay as described is exemplary and not intended to limit the scope of the invention. The skilled practitioner can appreciate that modifications including but not limited to RNA template, primer, nucleotides, NS5B polymerase form, buffer composition, can be made to develop similar assays that yield the same result for the efficacy of the compounds and compositions described in the invention.

NS5B polymerase inhibition data was calculated for the compounds of the present invention using this method and is set forth in the table below.

| Cpd. No. | IC₅₀ (μM) |
|---|---|
| 1 | 3 |
| 2 | 0.52 |
| 3 | 2 |
| 4 | >200 |
| 5 | >200 |
| 6 | 2.2 |
| 7 | 2.3 |
| 8 | 1 |
| 9 | 46 |
| 10 | 3 |
| 11 | >20 |
| 12 | 1 |
| 13 | 4.1 |
| 14 | 0.54 |
| 15 | 54 |
| 16 | 5.6 |
| 17 | 14 |
| 18 | 2.3 |
| 19 | 6.7 |
| 20 | 0.58 |
| 21 | 3 |
| 22 | 10 |
| 23 | 7 |
| 24 | >20 |
| 25 | 2.3 |
| 26 | >20 |
| 27 | 13 |
| 28 | 14 |
| 29 | 15 |
| 30 | 20 |
| 31 | >20 |
| 32 | >200 |
| 33 | >20 |
| 34 | >20 |
| 35 | >20 |
| 36 | >20 |
| 37 | >20 |
| 38 | 13 |
| 39 | >200 |
| 40 | 0.5 |
| 41 | 0.56 |
| 42 | 0.16 |
| 43 | 2.3 |
| 44 | 1.5 |
| 45 | 3.9 |
| 46 | 140 |
| 47 | 2.5 |
| 48 | 15 |
| 49 | 3.9 |
| 50 | 5 |
| 51 | 7.3 |
| 52 | >100 |
| 53 | >200 |
| 54 | >200 |
| 55 | >200 |
| 56 | >200 |
| 57 | 0.1 |
| 58 | 0.027 |
| 59 | 0.014 |
| 60 | 0.032 |
| 61 | 1 |
| 62 | 17 |
| 63 | 3 |
| 64 | 7 |
| 65 | >20 |
| 66 | 12 |
| 67 | 8 |
| 68 | >20 |
| 69 | >5 |
| 70 | 13 |

### Example 11

### Cell-Based HCV Replicon Assay

To measure cell-based anti-HCV activity of the a Compound of Formula (I), replicon cells were seeded at 5000 cells/well in 96-well collagen I-coated Nunc plates in the presence of the Compound of Formula (I). Various concentrations of a Compound of Formula (I), typically in 10 serial 2-fold dilutions, were added to the assay mixture, with the starting concentration ranging from 250 μM to 1 M. The final concentration of DMSO was 0.5%, fetal bovine serum was 5%, in the assay media. Cells were harvested on day 3 by the addition of 1× cell lysis buffer (Ambion cat #8721). The replicon RNA level was measured using real time PCR (Taqman assay). The amplicon was located in 5B. The PCR primers were: 5B.2F, ATGGACAGGCGCCCTGA (SEQ ID NO: 2); 5B.2R, TTGATGGGCAGCTTGGTTTC (SEQ ID NO: 3); the probe sequence was FAM-labeled CACGCCATGCGCTGCGG (SEQ ID NO: 4). GAPDH RNA was used as endogenous control and was amplified in the same reaction as NS5B (multiplex PCR) using primers and VIC-labeled probe recommended by the manufacturer (PE Applied Biosystem). The real-time RT-PCR reactions were run on ABI PRISM 7900HT Sequence Detection System using the following program: 48° C. for 30 minutes, 95° C. for 10 minutes, 40 cycles of 95° C. for 15 sec, 60° C. for 1 minute. The ΔCT values (CT_{5B}-CT_{GAPDH}) were plotted against the concentration of test compound and fitted to the sigmoid dose-response model using XLfit4 (MDL). EC₅₀ was defined as the concentration of inhibitor necessary to achieve ΔCT-1 over the projected baseline; EC₉₀ the concentration necessary to achieve ΔCT=3.2 over the baseline. Alternatively, to quantitate the absolute amount of replicon RNA, a standard curve was established by including serially diluted T7 transcripts of replicon RNA in the Taqman assay. All Taqman reagents were from PE Applied Biosystems. Such an assay procedure was described in detail in e.g. Malcolm et al., Antimicrobial Agents and Chemotherapy 50: 1013-1020 (2006).

The study of the HCV life cycle has been difficult due to the lack of a cell-culture system to support the HCV virus. To date, compounds in different structural classes acting on different sites within the HCV polyprotein have demonstrated efficacy in various species, including humans, in reducing HCV viral titers. Furthermore, the subgenomic replicon assay is highly correlated with efficacy in non-humans and humans infected with HCV. See K. del Carmen et al., Annals of Hepatology, 2004, 3:54.

It is accepted that the HCV replicon system described above is useful for the development and the evaluation of antiviral drugs. See Pietschmann, T. & Bartenschlager, R., Current Opinion in Drug Discovery Research 2001, 4:657-664).

HCV replicon assay data was calculated for the compounds of the present invention using this method and the compounds demonstrated EC₅₀ values between 1 μM and 50 μM.

### Uses of the 1,4-Substituted Piperazine Derivatives

The 1,4-Substituted Piperazine Derivatives are useful in human and veterinary medicine for treating or preventing a viral infection or a virus-related disorder in a patient. In accordance with the invention, the 1,4-Substituted Piperazine Derivatives can be administered to a patient in need of treatment or prevention of a viral infection or a virus-related disorder.

Accordingly, in one embodiment, the invention provides methods for treating a viral infection in a patient comprising administering to the patient an effective amount of one or more 1,4-Substituted Piperazine Derivatives or a pharmaceutically acceptable salt thereof. In another embodiment, the invention provides methods for treating a virus-related disorder in a patient comprising administering to the patient an effective amount of one or more 1,4-Substituted Piperazine Derivatives or a pharmaceutically acceptable salt thereof.

### Treatment or Prevention of a Viral Infection

The 1,4-Substituted Piperazine Derivatives can be used to treat or prevent a viral infection. In one embodiment, the 1,4-Substituted Piperazine Derivatives can be inhibitors of viral replication. In a specific embodiment, the 1,4-Substituted Piperazine Derivatives can be inhibitors of HCV replication. Accordingly, the 1,4-Substituted Piperazine Derivatives are useful for treating viral diseases and disorders related to the activity of a virus, such as HCV polymerase.

Examples of viral infections that can be treated or prevented using the present methods, include but are not limited to, hepatitis A infection, hepatitis B infection and hepatitis C infection.

In one embodiment, the viral infection is hepatitis C infection.

In one embodiment, the hepatitis C infection is acute hepatitis C. In another embodiment, the hepatitis C infection is chronic hepatitis C.

The compositions and combinations of the present invention can be useful for treating a patient suffering from infection related to any HCV genotype. HCV types and subtypes may differ in their antigenicity, level of viremia, severity of disease produced, and response to interferon therapy as described in Holland et al., Pathology, 30(2):192-195 (1998). The nomenclature set forth in Simmonds et al., J Gen Virol, 74(Pt11):2391-2399 (1993) is widely used and classifies isolates into six major genotypes, 1 through 6, with two or more related subtypes, e.g., 1a, 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3 (see Lamballerie et al, J Gen Viral, 78(Ptl):45-51 (1997)). The major genotypes have been defined as having sequence similarities of between 55 and 72% (mean 64.5%), and subtypes within types as having 75%-86% similarity (mean 80%) when sequenced in the NS-5 region (see Simmonds et al., J Gen Virol, 75(Pt 5):1053-1061 (1994)).

### Treatment or Prevention of a Virus-Related Disorder

The 1,4-Substituted Piperazine Derivatives can be used to treat or prevent a virus-related disorder. Accordingly, the 1,4-Substituted Piperazine Derivatives are useful for treating disorders related to the activity of a virus, such as liver inflammation or cirrhosis. Virus-related disorders include, but are not limited to, RNA-dependent polymerase-related disorders and disorders related to HCV infection.

### Treatment or Prevention of a RNA-Dependent Polymerase-Related Disorder

The 1,4-Substituted Piperazine Derivatives are useful for treating or preventing a RNA dependent polymerase (RdRp) related disorder in a patient. Such disorders include viral infections wherein the infective virus contains a RdRp enzyme.

Accordingly, in one embodiment, the present invention provides a method for treating a RNA dependent polymerase-related disorder in a patient, comprising administering to the patient an effective amount of one or more 1,4-Substituted Piperazine Derivatives or a pharmaceutically acceptable salt thereof.

### Treatment or Prevention of a Disorder Related to HCV Infection

The 1,4-Substituted Piperazine Derivatives can also be useful for treating or preventing a disorder related to an HCV infection. Examples of such disorders include, but are not limited to, cirrhosis, portal hypertension, ascites, bone pain, varices, jaundice, hepatic encephalopathy, thyroiditis, porphyria cutanea tarda, cryoglobulinemia, glomerulonephritis, sicca syndrome, thrombocytopenia, lichen planus and diabetes mellitus.

Accordingly, in one embodiment, the invention provides methods for treating an HCV-related disorder in a patient, wherein the method comprises administering to the patient a therapeutically effective amount of one or more 1,4-Substituted Piperazine Derivatives, or a pharmaceutically acceptable salt thereof.

### Combination Therapy

In another embodiment, the present methods for treating or preventing a viral infection or a virus-related disorder can further comprise the administration of one or more additional therapeutic agents which are not 1,4-Substituted Piperazine Derivatives.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a patient, the method comprising administering to the patient: (i) one or more 1,4-Substituted Piperazine Derivatives, or a pharmaceutically acceptable salt thereof, and (ii) at least one additional therapeutic agent that is other than a 1,4-Substituted Piperazine Derivative, wherein the amounts administered are together effective to treat or prevent a viral infection.

When administering a combination therapy of the invention to a patient, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a 1,4-Substituted Piperazine Derivative and an additional therapeutic agent may be present in fixed amounts (dosage amounts) in a single dosage unit (e.g., a capsule, a tablet and the like). A commercial example of such single dosage unit containing fixed amounts of two different active compounds is VYTORIN® (available from Merck Schering-Plough Pharmaceuticals, Kenilworth, N.J.).

In one embodiment, the one or more 1,4-Substituted Piperazine Derivatives is administered during a time when the additional therapeutic agent(s) exert their prophylactic or therapeutic effect, or vice versa.

In another embodiment, the one or more 1,4-Substituted Piperazine Derivatives and the additional therapeutic agent(s) are administered in doses commonly employed when such agents are used as monotherapy for treating a viral infection.

In another embodiment, the one or more 1,4-Substituted Piperazine Derivatives and the additional therapeutic agent(s) are administered in doses lower than the doses commonly employed when such agents are used as monotherapy for treating a viral infection.

In still another embodiment, the one or more 1,4-Substituted Piperazine Derivatives and the additional therapeutic agent(s) act synergistically and are administered in doses lower than the doses commonly employed when such agents are used as monotherapy for treating a viral infection.

In one embodiment, the one or more 1,4-Substituted Piperazine Derivatives and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HCV infection.

The one or more 1,4-Substituted Piperazine Derivatives and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the admithstration of one or more 1,4-Substituted Piperazine Derivatives and the additional⁻therapeutic agent(s) may inhibit the resistance of a viral infection to these agents.

Non-limiting examples of additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, an antibody therapy (monoclonal or polyclonal), and any agent useful for treating an RNA-dependent polymerase-related disorder. In one embodiment, additional therapeutic agents useful in the present compositions and methods include an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, and an antibody therapy (monoclonal or polyclonal).

In one embodiment, the additional therapeutic agent is a viral protease inhibitor.

In another embodiment, the additional therapeutic agent is a viral replication inhibitor.

In another embodiment, the additional therapeutic agent is an HCV NS3 protease inhibitor.

In another embodiment, the additional therapeutic agent is an HCV NS5B polymerase inhibitor.

In another embodiment, the additional therapeutic agent is a nucleoside inhibitor.

In another embodiment, the additional therapeutic agent is an interferon.

In one embodiment, the additional therapeutic agent is an HCV replicase inhibitor.

In another embodiment, the additional therapeutic agent is an antisense agent.

In another embodiment, the additional therapeutic agent is a therapeutic vaccine.

In a further embodiment, the additional therapeutic agent is a virion production inhibitor.

In another embodiment, the additional therapeutic agent is an antibody therapy.

In another embodiment, the additional therapeutic agent is an HCV NS2 inhibitor.

In another embodiment, the additional therapeutic agent is an HCV NS4A inhibitor.

In another embodiment, the additional therapeutic agent is an HCV NS4B inhibitor.

In another embodiment, the additional therapeutic agent is an HCV NS5A inhibitor

In another embodiment, the additional therapeutic agent is an HCV NS3 helicase inhibitor.

In another embodiment, the additional therapeutic agent is an HCV IRES inhibitor.

In another embodiment, the additional therapeutic agent is an HCV p7 inhibitor.

In another embodiment, the additional therapeutic agent is an HCV entry inhibitor.

In another embodiment, the additional therapeutic agent is an HCV assembly inhibitor.

In one embodiment, the additional therapeutic agents comprise a protease inhibitor and a polymerase inhibitor.

In still another embodiment, the additional therapeutic agents comprise a protease inhibitor and an immunomodulatory agent.

In yet another embodiment, the additional therapeutic agents comprise a polymerase inhibitor and an immunomodulatory agent.

In another embodiment, the additional therapeutic agents comprise a protease inhibitor and a nucleoside.

In another embodiment, the additional therapeutic agents comprise an immunomodulatory agent and a nucleoside.

In one embodiment, the additional therapeutic agents comprise a protease inhibitor and a NS5A inhibitor.

In another embodiment, the additional therapeutic agents comprise a nucleoside and a NS5A inhibitor.

In another embodiment, the additional therapeutic agents comprise a protease inhibitor, an immunomodulatory agent and a nucleoside.

In still another embodiment, the additional therapeutic agents comprise a protease inhibitor, a nucleoside and a NS5A inhibitor.

In a further embodiment, the additional therapeutic agents comprise a protease inhibitor, a polymerase inhibitor and an immunomodulatory agent.

In another embodiment, the additional therapeutic agent is ribavirin.

HCV polymerase inhibitors useful in the present compositions and methods include, but are not limited to, VP-19744 (Wyeth/ViroPharma), PSI-7851 (Pharmasset), R7128 (Roche/Pharmasset), PF-868554/filibuvir (Pfizer), VCH-759 (ViroChem Pharma), HCV-796 (Wyeth/ViroPharma), IDX-184 (Idenix), IDX-375 (Idenix), NM-283 (Idenix/Novartis), R-1626 (Roche), MK-0608 (Isis/Merck), INX-8014 (Inhibitex), INX-8018 (Inhibitex), INX-189 (Inhibitex), GS 9190 (Gilead), A-848837 (Abbott), ABT-333 (Abbott), ABT-072 (Abbott), A-837093 (Abbott), BI-207127 (Boehringer-Ingelheim), BILB-1941 (Boehringer-Ingelheim), MK-3281 (Merck), VCH222 (ViroChem), VCH916 (ViroChem), VCH716 (ViroChem), GSK-71185 (Glaxo SmithKline), ANA598 (Anadys), GSK-625433 (Glaxo SmithKline), XTL-2125 (XTL Biopharmaceuticals), and those disclosed in Ni et al., Current Opinion in Drug Discovery and Development, 7(4):446 (2004); Tan et al., Nature Reviews, 1:867 (2002); and Beaulieu et al., Current Opinion in Investigational Drugs, 5:838 (2004).

Other HCV polymerase inhibitors useful in the present compositions and methods include, but are not limited to, those disclosed in International Publication Nos. WO 08/082484, WO 08/082488, WO 08/083351, WO 08/136815, WO 09/032116, WO 09/032123, WO 09/032124 and WO 09/032125.

Interferons useful in the present compositions and methods include, but are not limited to, interferon alfa-2a, interferon alfa-2b, interferon alfacon-1 and PEG-interferon alpha conjugates. “PEG-interferon alpha conjugates” are interferon alpha molecules covalently attached to a PEG molecule. Illustrative PEG-interferon alpha conjugates include interferon alpha-2a (Roferon™, Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™ from Schering-Plough Corporation), interferon alpha-2b-XL (e.g., as sold under the trade name PEG-Intron™), interferon alpha-2c (Berofor Alpha™, Boehringer Ingelheim, Ingelheim, Germany), PEG-interferon lambda (Bristol-Myers Squibb and ZymoGenetics), interferon alfa-2b alpha fusion polypeptides, interferon fused with the human blood protein albumin (Albuferon™, Human Genome Sciences), Omega Interferon (Intarcia), Locteron controlled release interferon (Biolex/OctoPlus), Biomed-510 (omega interferon), Peg-IL-29 (ZymoGenetics), Locteron CR (Octoplus), IFN-α-2b-XL (Flamel Technologies), and consensus interferon as defined by determination of a consensus sequence of naturally occurring interferon alphas (Infergen™, Amgen, Thousand Oaks, Calif).

Antibody therapy agents useful in the present compositions and methods include, but are not limited to, antibodies specific to IL-10 (such as those disclosed in US Patent Publication No. US2005/0101770, humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like).

Examples of viral protease inhbitors useful in the present compositions and methods include, but are not limited to, a HCV protease inhibitor.

HCV protease inhibitors useful in the present compositions and methods include, but are not limited to, those disclosed in U.S. Pat. Nos. 7,494,988, 7,485,625, 7,449,447, 7,442,695, 7,425,576, 7,342,041, 7,253,160, 7,244,721, 7,205,330, 7,192,957, 7,186,747, 7,173,057, 7,169,760, 7,012,066, 6,914,122, 6,911,428, 6,894,072, 6,846,802, 6,838,475, 6,800,434, 6,767,991, 5,017,380, 4,933,443, 4,812,561 and 4,634,697; U.S. Patent Publication Nos. US20020068702, US20020160962, US20050119168, US20050176648, US20050209164, US20050249702 and US20070042968; and International Publication Nos. WO 03/006490, WO 03/087092, WO 04/092161 and WO 08/124148.

Additional HCV protease inhibitors useful in the present compositions and methods include, but are not limited to, SCHSO3034 (Boceprevir, Schering-Plough), SCH900518 (Schering-Plough), VX-950 (Telaprevir, Vertex), VX-500 (Vertex), VX-813 (Vertex), VBY-376 (Virobay), BI-201335 (Boehringer Ingelheim), TMC-435 (Medivir/Tibotec), ABT-450 (Abbott), MK-7009 (Merck), TMC-435350 (Medivir), ITMN-191/R7227 (InterMune/Roche), EA-058 (Abbott/Enanta), EA-063 (Abbott/Enanta), GS-9132 (Gilead/Achillion), ACH-1095 (Gilead/Achillon), IDX-136 (Idenix), IDX-316 (Idenix), ITMN-8356 (InterMune), ITMN-8347 (InterMune), ITMN-8096 (InterMune), ITMN-7587 (InterMune), PHX1766 (Phenomix), amprenavir, atazanavir, fosemprenavir, indinavir, lopinavir, ritonavir, nelfinavir, saquinavir, tipranavir, Kaletra (a combination of ritonavir and lopinavir) and TMC114.

Additional examples of HCV protease inhbitors useful in the present compositions and methods include, but are not limited to, those disclosed in Landro et al., Biochemistry, 36(30:9340-9348 (1997); Ingallinella et al., Biochemistry, 37(25):8906-8914 (1998); Llinàs-Brunet et al., Bioorg Med Chem Lett, 8(13):1713-1718 (1998); Martin et al., Biochemistry, 37(33):11459-11468 (1998); Dimasi et al., J Virol, 71(10):7461-7469 (1997); Martin et al., Protein Eng, 10(5):607-614 (1997); Elzouki et al., J Hepat, 27(1):42-48 (1997); BioWorld Today, 9(217):4 (Nov. 10, 1998); U.S. Patent Publication Nos. US2005/0249702 and US 2007/0274951; and International Publication Nos. WO 98/14181, WO 98/17679, WO 98/17679, WO 98/22496 and WO 99/07734 and WO 05/087731.

Further examples of HCV protease inhibitors useful in the present compositions and methods include, but are not limited to, the following compounds:

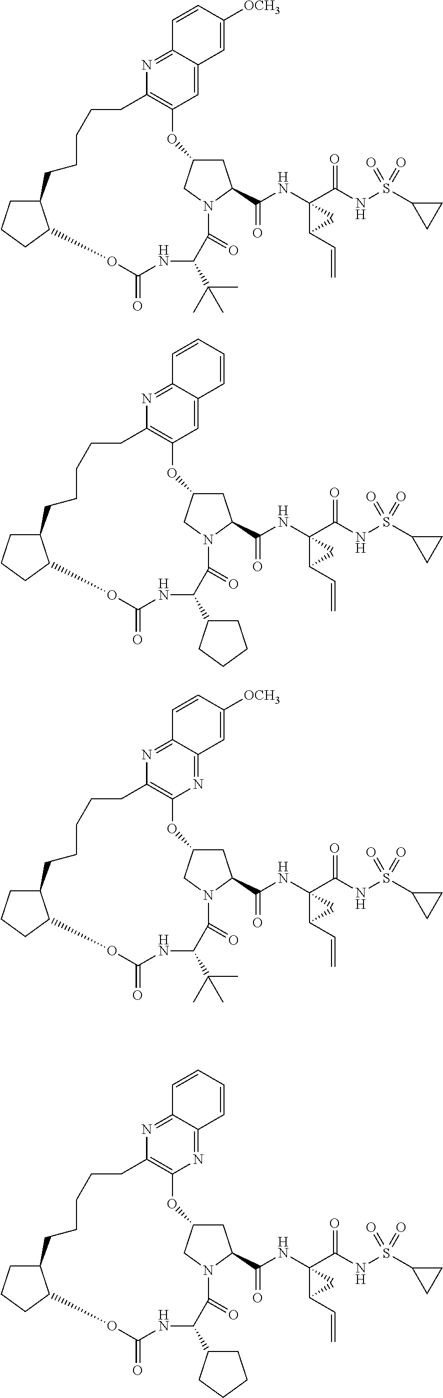

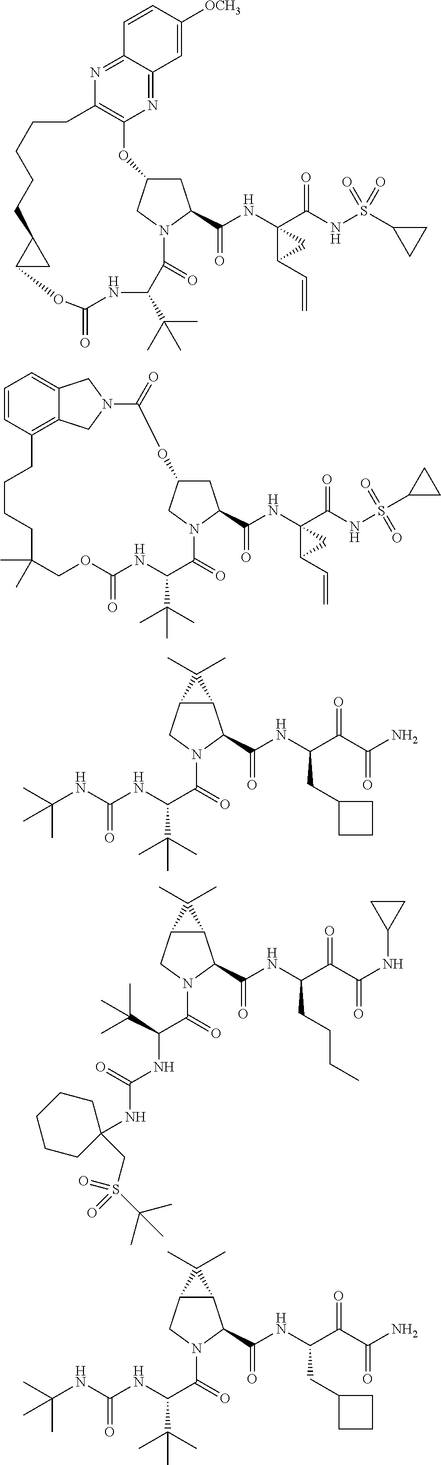

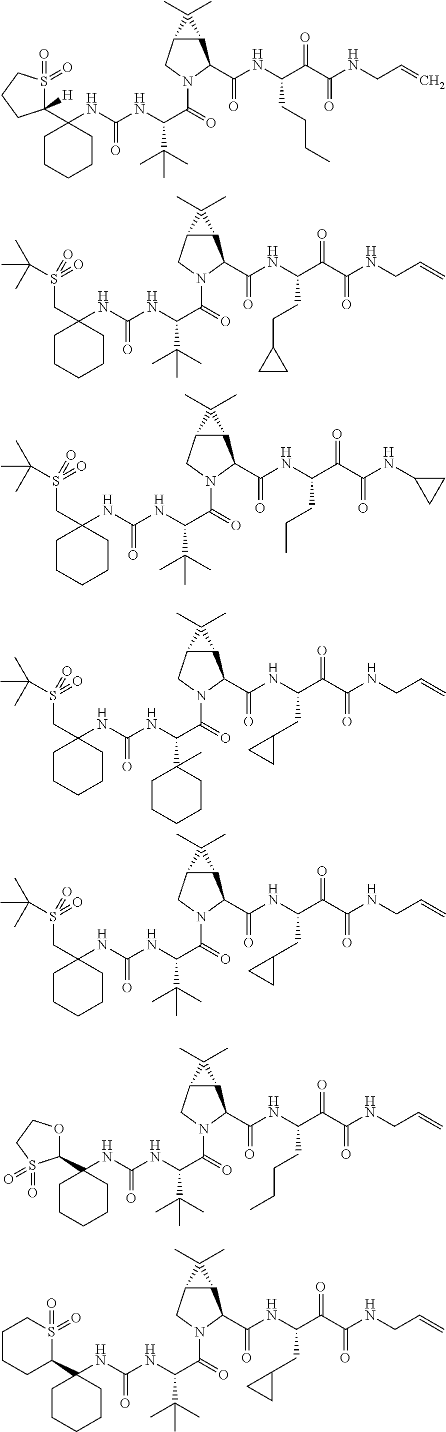

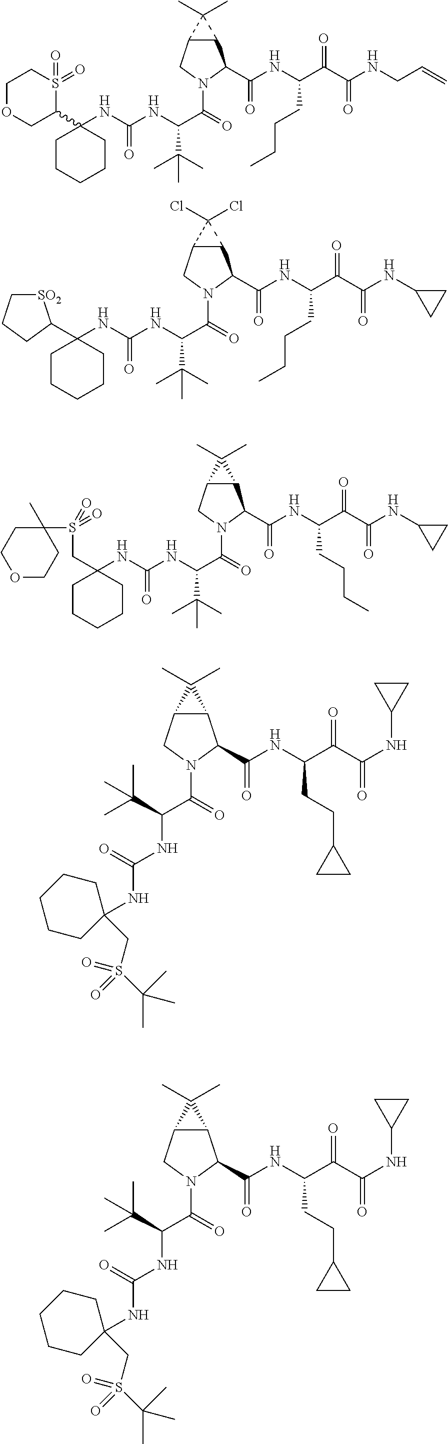

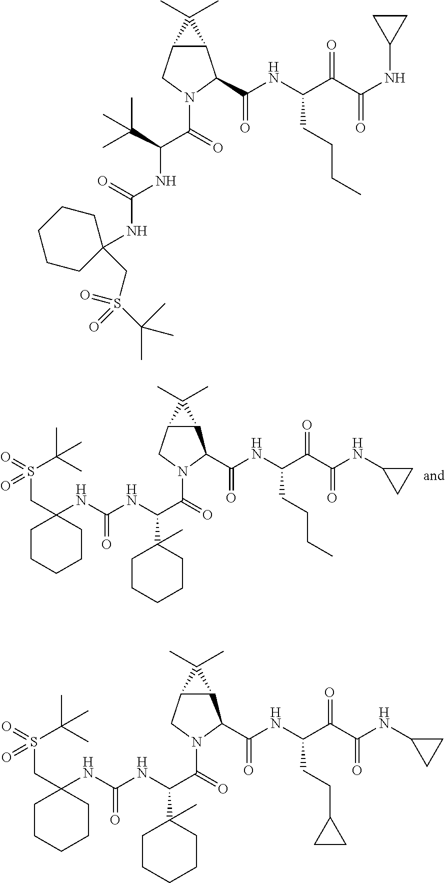

and pharmaceutically acceptable salts thereof.

Viral replication inhibitors useful in the present compositions and methods include, but are not limited to, HCV replicase inhibitors, IRES inhibitors, NS4A inhibitors, NS3 helicase inhibitors, NS5A inhibitors, ribavirin, AZD-2836 (Astra Zeneca), BMS-790052 (Bristol-Myers Squibb), viramidine, A-831 (Arrow Therapeutics); an antisense agent or a therapeutic vaccine.

In one embodiment, viral replication inhibitors useful in the present compositions and methods include, but are not limited to, HCV replicase inhibitors, IRES inhibitors, NS4A inhibitors, NS3 helicase inhibitors and NS5A inhibitors.

HCV NS4A inhibitors useful in the useful in the present compositions and methods include, but are not limited to, those disclosed in U.S. Pat. Nos. 7,476,686 and 7,273,885; U.S. Patent Publication No. US20090022688; and International Publication Nos. WO 2006/019831 and WO 2006/019832. Additional HCV NS4A inhibitors useful in the useful in the present compositions and methods include, but are not limited to, AZD2836 (Astra Zeneca) and ACH-806 (Achillon Pharmaceuticals, New Haven, Conn.).

HCV replicase inhibitors useful in the useful in the present compositions and methods include, but are not limited to, those disclosed in U.S. Patent Publication No. US20090081636.

Therapeutic vaccines useful in the present compositions and methods include, but are not limited to, IC41 (Intercell Novartis), CSL123 (Chiron/CSL), GI 5005 (Globeimmune), TG-4040 (Transgene), GNI-103 (GENimmune), Hepavaxx C (ViRex Medical), ChronVac-C (Inovio/Tripep), PeviPROTM (Pevion Biotect), HCV/MF59 (Chiron/Novartis) and Civacir (NABI).

Examples of further additional therapeutic agents useful in the present compositions and methods include, but are not limited to, TT033 (Benitec/Tacere Bio/Pfizer), Sirna-034 (Sirna Therapeutics), GNI-104 (GENimmune), GI-5005 (GlobeImmune), IDX-102 (Idenix), Levovirin™ (ICN Pharmaceuticals, Costa Mesa, Calif.); Humax (Genmab), ITX-2155 (Ithrex/Novartis), PRO 206 (Progenies), HepaCide-I (NanoVirocides), MX3235 (Migenix), SCY-635 (Scynexis); KPE02003002 (Kemin Pharma), Lenocta (VioQuest Pharmaceuticals), IET—Interferon Enhancing Therapy (Transition Therapeutics), Zadaxin (SciClone Pharma), VP 50406™ (Viropharma, Incorporated, Exton, Pa.); Taribavirin (Valeant Pharmaceuticals); Nitazoxanide (Romark); Debio 025 (Debiopharm); GS-9450 (Gilead); PF-4878691 (Pfizer); ANA773 (Anadyr); SCV-07 (SciClone Pharmaceuticals); NIM-881 (Novartis); ISIS 14803™ (ISIS Phannaceuticals, Carlsbad, Calif.); Heptazyme™ (Ribozyme Pharmaceuticals, Boulder, Colo.); Thymosin™ (SciClone Pharmaceuticals, San Mateo, Calif.); Maxamine™ (Maxim Pharmaceuticals, San Diego, Calif.); NKB-122 (JenKen Bioscience Inc., North Carolina); Alinia (Romark Laboratories), INFORM-1 (a combination of R7128 and ITMN-191); and mycophenolate mofetil (Hoffman-LaRoche, Nutley, N.J.).

The doses and dosage regimen of the other agents used in the combination therapies of the present invention for the treatment or prevention of a viral infection or virus-related disorder can be determined by the attending clinician, taking into consideration the approved doses and dosage regimen in the package insert; the age, sex and general health of the patient; and the type and severity of the viral infection or related disease or disorder. When administered in combination, the 1,4-Substituted Piperazine Derivative(s) and the other agent(s) can be administered simultaneously (i.e., in the same composition or in separate compositions one right after the other) or sequentially. This is particularly useful when the components of the combination are given on different dosing schedules, e.g., one component is administered once daily and another every six hours, or when the preferred pharmaceutical compositions are different, e.g., one is a tablet and one is a capsule. A kit comprising the separate dosage forms is therefore advantageous.

Generally, a total daily dosage of the one or more 1,4-Substituted Piperazine Derivatives(s) alone, or when administered as combination therapy, can range from about 1 to about 2500 mg per day, although variations will necessarily occur depending on the target of therapy, the patient and the route of administration. In one embodiment, the dosage is from about 10 to about 1000 mg/day, administered in a single dose or in 2-4 divided doses. In another embodiment, the dosage is from about 1 to about 500 mg/day, administered in a single dose or in 2-4 divided doses. In still another embodiment, the dosage is from about 1 to about 100 mg/day, administered in a single dose or in 2-4 divided doses. In yet another embodiment, the dosage is from about 1 to about 50 mg/day, administered in a single dose or in 2-4 divided doses. In another embodiment, the dosage is from about 500 to about 1500 mg/day, administered in a single dose or in 2-4 divided doses. In still another embodiment, the dosage is from about 500 to about 1000 mg/day, administered in a single dose or in 2-4 divided doses. In yet another embodiment, the dosage is from about 100 to about 500 mg/day, administered in a single dose or in 2-4 divided doses.

In one embodiment, when the additional therapeutic agent is INTRON-A interferon alpha 2b (commercially available from Schering-Plough Corp.), this agent is administered by subcutaneous injection at 3MIU (12 mcg)/0.5 mL/TIW for 24 weeks or 48 weeks for first time treatment.

In another embodiment, when the additional therapeutic agent is PEG-INTRON interferon alpha 2b pegylated (commercially available from Schering-Plough Corp.), this agent is administered by subcutaneous injection at 1.5 mcg/kg/week, within a range of 40 to 150 mcg/week, for at least 24 weeks.

In another embodiment, when the additional therapeutic agent is ROFERON A inteferon alpha 2a (commercially available from Hoffmann-La Roche), this agent is administered by subcutaneous or intramuscular injection at 3MIU (11.1 mcg/mL)/TIW for at least 48 to 52 weeks, or alternatively 6MIU/TIW for 12 weeks followed by 3MIU/TIW for 36 weeks.

In still another embodiment, when the additional therapeutic agent is PEGASUS interferon alpha 2a pegylated (commercially available from Hoffmann-La Roche), this agent is administered by subcutaneous injection at 180 mcg/1 mL or 180 mcg/0.5 mL, once a week for at least 24 weeks.

In yet another embodiment, when the additional therapeutic agent is INFERGEN interferon alphacon-1 (commercially available from Amgen), this agent is administered by subcutaneous injection at 9 mcg/TIW is 24 weeks for first time treatment and up to 15 mcg/TIW for 24 weeks for non-responsive or relapse treatment.

In a further embodiment, when the additional therapeutic agent is Ribavirin (commercially available as REBETOL ribavirin from Schering-Plough or COPEGUS ribavirin from Hoffmann-La Roche), this agent is administered at a daily dosage of from about 600 to about 1400 mg/day for at least 24 weeks.

In one embodiment, one or more compounds of the present invention are administered with one or more additional therapeutic agents selected from a HCV protease inhibitor, a HCV replication inhibitor, a nucleoside, an interferon, a pegylated interferon and ribavirin. The combination therapies can include any combination of these additional therapeutic agents.

In another embodiment, one or more compounds of the present invention are administered with one additional therapeutic agent selected from a HCV protease inhibitor, a HCV replication inhibitor, a nucleoside, an interferon, a pegylated interferon and ribavirin.

In another embodiment, one or more compounds of the present invention are administered with two additional therapeutic agents selected from a HCV protease inhibitor, a HCV replication inhibitor, a nucleoside, an interferon, a pegylated interferon and ribavirin.

In a specific embodiment, one or more compounds of the present invention are administered with a HCV protease inhibitor and ribavirin. In another specific embodiment, one or more compounds of the present invention are administered with a pegylated interferon and ribavirin.

In another embodiment, one or more compounds of the present invention are administered with three additional therapeutic agents selected from a HCV protease inhibitor, a HCV replication inhibitor, a nucleoside, an interferon, a pegylated interferon and ribavirin.

In one embodiment, one or more compounds of the present invention are administered with one or more additional therapeutic agents selected from a HCV polymerase inhibitor, a viral protease inhibitor, an interferon, and a viral replication inhibitor. In another embodiment, one or more compounds of the present invention are administered with one or more additional therapeutic agents selected from a HCV polymerase inhibitor, a viral protease inhibitor, an interferon, and a viral replication inhibitor. In another embodiment, one or more compounds of the present invention are administered with one or more additional therapeutic agents selected from a HCV polymerase inhibitor, a viral protease inhibitor, an interferon, and ribavirin.

In one embodiment, one or more compounds of the present invention are administered with one additional therapeutic agent selected from a HCV polymerase inhibitor, a viral protease inhibitor, an interferon, and a viral replication inhibitor. In another embodiment, one or more compounds of the present invention are administered with ribavirin.

In one embodiment, one or more compounds of the present invention are administered with two additional therapeutic agents selected from a HCV polymerase inhibitor, a viral protease inhibitor, an interferon, and a viral replication inhibitor.

In another embodiment, one or more compounds of the present invention are administered with ribavirin, interferon and another therapeutic agent.

In another embodiment, one or more compounds of the present invention are administered with ribavirin, interferon and another therapeutic agent, wherein the additional therapeutic agent is selected from a HCV polymerase inhibitor, a viral protease inhibitor, and a viral replication inhibitor.

In still another embodiment, one or more compounds of the present invention are administered with ribavirin, interferon and a viral protease inhibitor.

In another embodiment, one or more compounds of the present invention are administered with ribavirin, interferon and an HCV protease inhibitor.

In another embodiment, one or more compounds of the present invention are administered with ribavirin, interferon and boceprevir or telaprevir.

In a further embodiment, one or more compounds of the present invention are administered with ribavirin, interferon and an HCV polymerase inhibitor.

### Compositions and Administration

Due to their activity, the 1,4-Substituted Piperazine Derivatives are useful in veterinary and human medicine. As described above, the 1,4-Substituted Piperazine Derivatives are useful for treating or preventing a viral infection or a virus-related disorder in a patient in need thereof.

When administered to a patient, the 1,4-Substituted Piperazine Derivatives can be administered as a component of a composition that comprises a pharmaceutically acceptable carrier or vehicle. The present invention provides pharmaceutical compositions comprising an effective amount of one or more 1,4-Substituted Piperazine Derivatives and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

The 1,4-Substituted Piperazine Derivatives of the present invention may also be deliverable transdermally. The transdermal compositions can take the form of creams, lotions, aerosols and/or emulsions and can be included in a transdermal patch of the matrix or reservoir type as are conventional in the art for this purpose.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more 1,4-Substituted Piperazine Derivatives are administered orally.

In another embodiment, the one or more 1,4-Substituted Piperazine Derivatives are administered intravenously.

In another embodiment, the one or more 1,4-Substituted Piperazine Derivatives are administered topically.

In still another embodiment, the one or more 1,4-Substituted Piperazine Derivatives are administered sublingually.

In one embodiment, a pharmaceutical preparation comprising one or more 1,4-Substituted Piperazine Derivatives is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the 1,4-Substituted Piperazine Derivative(s) by weight or volume. In various embodiments, the present compositions can contain, in one embodiment, from about 1% to about 70% or from about 5% to about 60% of the 1,4-Substituted Piperazine Derivative(s) by weight or volume.

The quantity of 1,4-Substituted Piperazine Derivative in a unit dose of preparation may be varied or adjusted from about 1 mg to about 2500 mg. In various embodiment, the quantity is from about 10 mg to about 1000 mg, 1 mg to about 500 mg, 1 mg to about 100 mg, and 1 mg to about 100 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the 1,4-Substituted Piperazine Derivatives will be regulated according to the judgment of the attending clinician considering such factors as age, condition and size of the patient as well as severity of the symptoms being treated. Generally, a total daily dosage of the 1,4-Substituted Piperazine Derivatives range from about 0.1 to about 2000 mg per day, although variations will necessarily occur depending on the target of therapy, the patient and the route of administration. In one embodiment, the dosage is from about 1 to about 200 mg/day, administered in a single dose or in 2-4 divided doses. In another embodiment, the dosage is from about 10 to about 2000 mg/day, administered in a single dose or in 2-4 divided doses. In another embodiment, the dosage is from about 100 to about 2000 mg/day, administered in a single dose or in 2-4 divided doses. In still another embodiment, the dosage is from about 500 to about 2000 mg/day, administered in a single dose or in 2-4 divided doses.

The compositions of the invention can further comprise one or more additional therapeutic agents, selected from those listed above herein. Accordingly, in one embodiment, the present invention provides compositions comprising: (i) one or more 1,4-Substituted Piperazine Derivatives or a pharmaceutically acceptable salt thereof; (ii) one or more additional therapeutic agents that are not a 1,4-Substituted Piperazine Derivative; and (iii) a pharmaceutically acceptable carrier, wherein the amounts in the composition are together effective to treat a viral infection or a virus-related disorder.

### Kits

In one aspect, the present invention provides a kit comprising a therapeutically effective amount of one or more 1,4-Substituted Piperazine Derivatives, or a pharmaceutically acceptable salt, solvate, ester or prodrug of said compound and a pharmaceutically acceptable carrier, vehicle or diluent.

In another aspect the present invention provides a kit comprising an amount of one or more 1,4-Substituted Piperazine Derivatives, or a pharmaceutically acceptable salt, solvate, ester or prodrug of said compound and an amount of at least one additional therapeutic agent listed above, wherein the amounts of the two or more active ingredients result in a desired therapeutic effect. In one embodiment, the one or more 1,4-Substituted Piperazine Derivatives and the one or more additional therapeutic agents are provided in the same container. In one embodiment, the one or more 1,4-Substituted Piperazine Derivatives and the one or more additional therapeutic agents are provided in separate containers.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula: 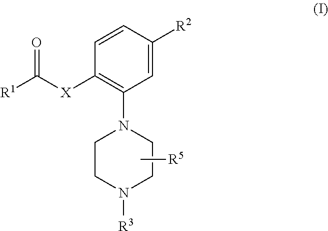
or a pharmaceutically acceptable salt thereof,
wherein:
X is —O—, —NH— or —N(alkyl)-;
R¹ is aryl, cycloalkyl, heteroaryl or heterocycloalkyl, wherein said aryl group, said cycloalkyl group, said heteroaryl group or said heterocycloalkyl group can be optionally substituted with up to 3 substituents, which can be the same or different, and are selected from alkyl, alkenyl, alkynyl, cycloalkyl, aryl, halo, haloalkyl, —N(R⁴)(R⁴), —OR⁴, —S(O)₂-alkyl, —NHC(O)R⁴, —C(O)N(R⁴)(R⁴) or —C(O)OR⁴;
R² is alkyl, halo, haloalkyl, alkenyl, alkynyl, cycloalkyl, aryl, heteroaryl, heterocycloalkyl, —C(O)OR⁴, —N(R⁴)(R⁴), —OR⁴, —S(O)₂-alkyl, —NHC(O)R⁴ or —C(O)N(R⁴)(R⁴);
R³ is aryl, heteroaryl, —S(O)₂-alkyl, —C(O)N(R⁴)(R⁴), —C(O)-alkylene-N(R⁴)(R⁴) or —C(O)OR⁴, wherein said aryl group or said heteroaryl group can be optionally substituted with up to 3 substituents, which can be the same or different, and are selected from alkyl, aryl, cycloalkyl, heterocycloalkyl, heteroaryl, halo, —alkylene-S-alkyl, alkenyl, alkynyl, —N(R⁴)(R⁴), —OR⁴, —S(O)₂-alkyl, —NHC(O)R⁴, —C(O)N(R⁴)(R⁴) or —C(O)OR⁴;
each occurrence of R⁴ is independently H, alkyl, aryl, cycloalkyl, heterocycloalkyl or heteroaryl; and
R⁵ represents one or more optional piperazine ring carbon substituents, which can be the same or different, and are selected from alkyl, cycloalkyl, halo, haloalkyl, —N(R⁴)(R⁴), —OR⁴, —S(O)₂-alkyl, —NHC(O)R⁴, —C(O)N(R⁴)(R⁴) or —C(O)OR⁴.

2. The compound of claim 1, wherein X is —NH—.

3. The compound of claim 1, wherein R¹ is phenyl, heteroaryl or heterocycloalkyl.

4. The compound of claim 1, wherein R¹ is:

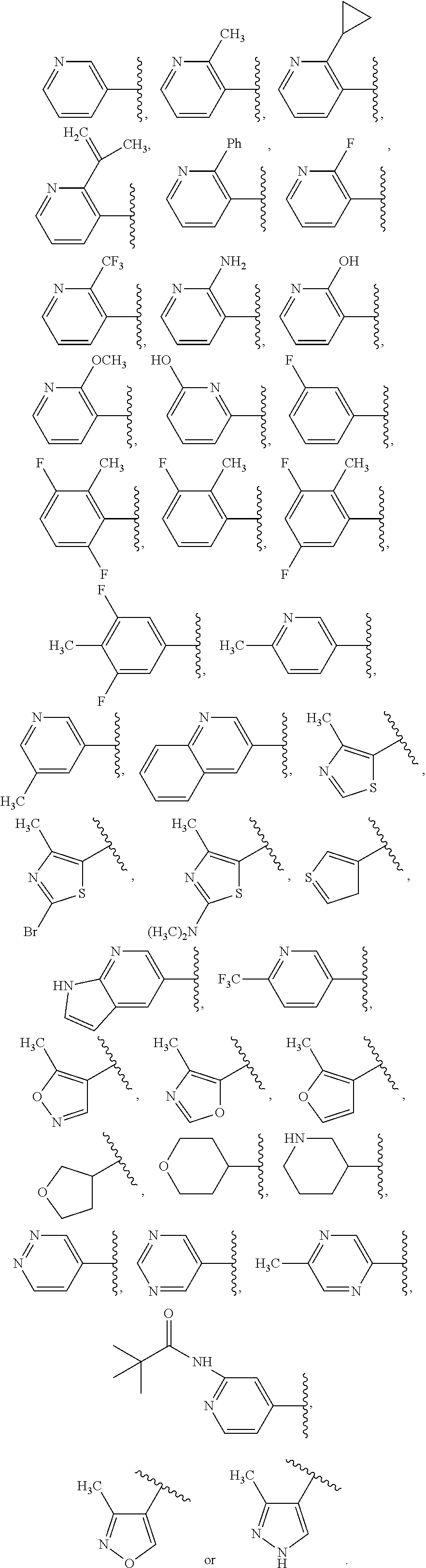

5. The compound of claim 1, wherein R² is alkyl.

6. The compound of claim 1, wherein R³ is heteroaryl.

7. The compound of claim 1, wherein R³ is

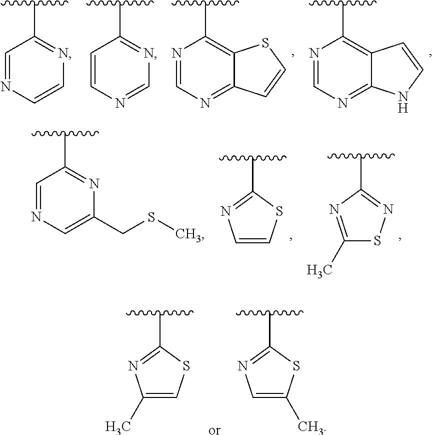

8. The compound of claim 1, wherein R¹ is 2-methyl pyridyl or 3,5-difluorobenzoyl

9. The compound of claim 1, wherein R² is alkyl.

10. The compound of claim 1, having the formula: 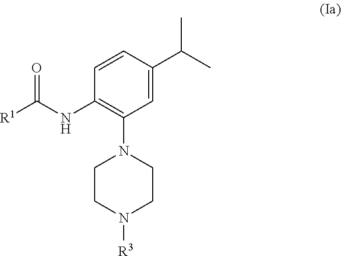
or a pharmaceutically acceptable salt thereof,
wherein:
R¹ is phenyl or pyridyl, wherein said phenyl group or said pyridyl group can be optionally substituted with up to 3 substituents, which can be the same or different, and are selected from alkyl, alkenyl, alkynyl, cycloalkyl, aryl, halo, haloalkyl, —N(R⁴)(R⁴), —OR⁴, —S(O)₂-alkyl, —NHC(O)R⁴, —C(O)N(R⁴)(R⁴) or —C(O)OR⁴;
R³ is phenyl, 5- or 6-membered heteroaryl, or —C(O)O-alkyl, wherein said phenyl group or said 5- or 6-membered heteroaryl group can be optionally substituted with up to 3 substituents, which can be the same or different, and are selected from alkyl, aryl, cycloalkyl, heterocycloalkyl, heteroaryl, halo, -alkylene-S-alkyl, alkenyl, alkynyl, —N(R⁴)(R⁴), —OR⁴, —S(O)₂-alkyl, —NHC(O)R⁴, —C(O)N(R⁴)(R⁴) or —C(O)OR⁴; and
each occurrence of R⁴ is independently H, alkyl, aryl, cycloalkyl, heterocycloalkyl or heteroaryl.

11. The compound of claim 10, wherein R¹ is: 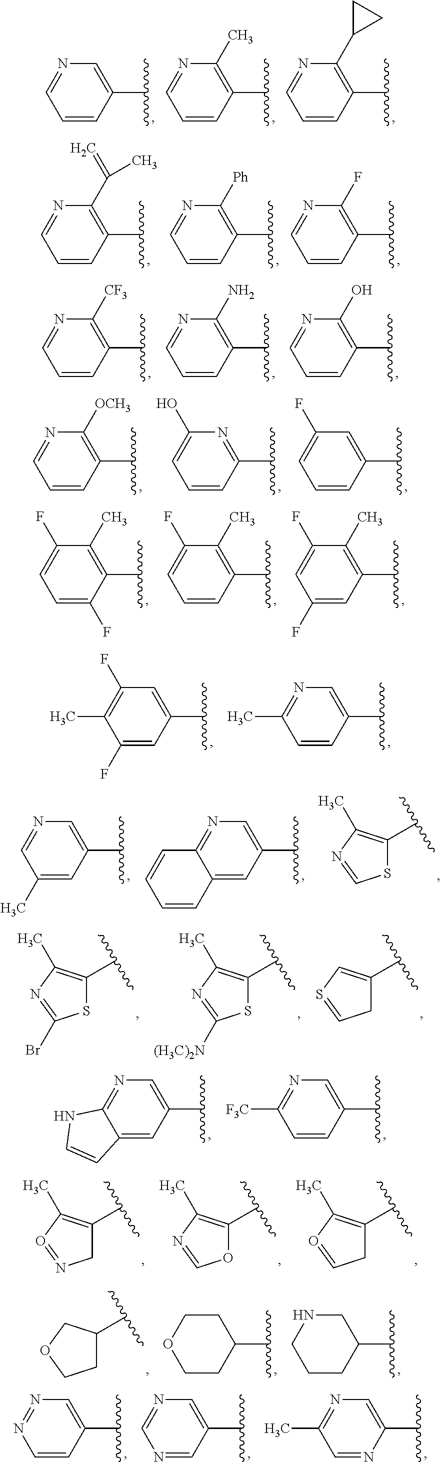 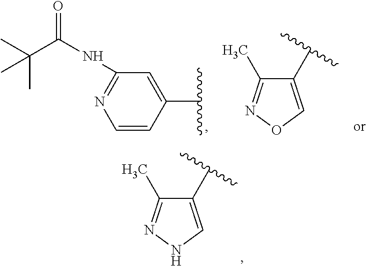
and R³ is:

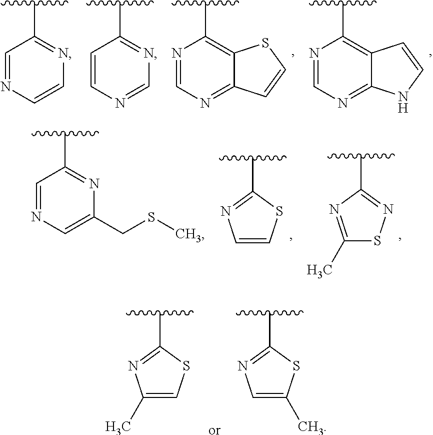

12. A compound having the structure: 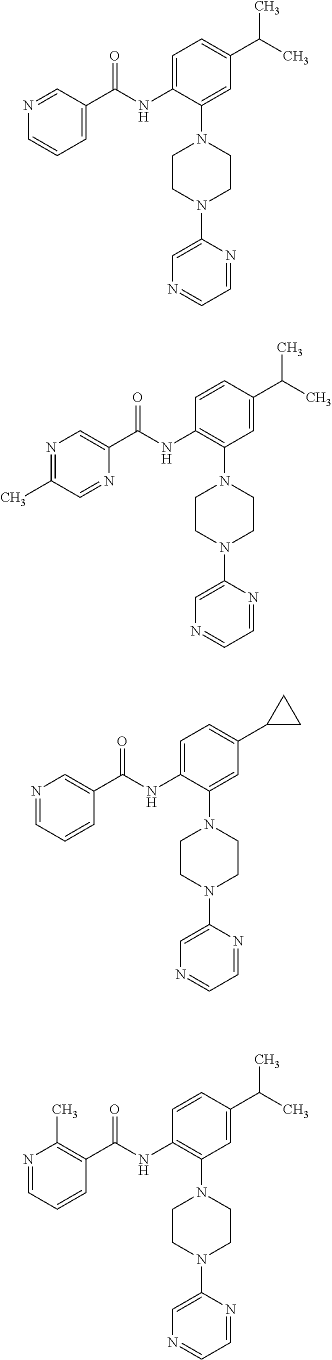 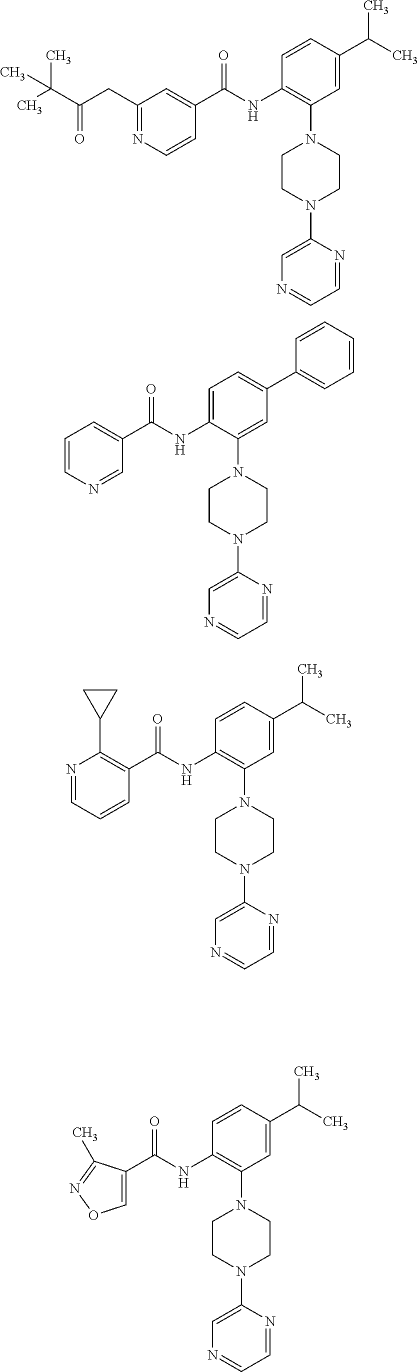 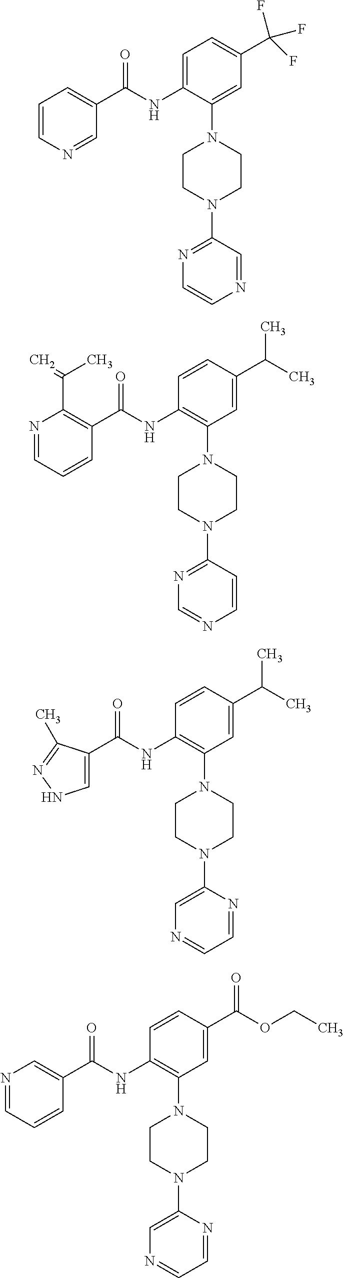 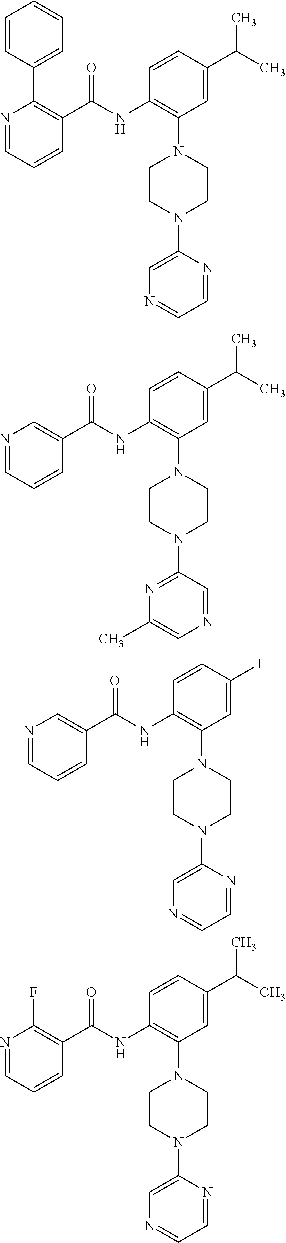 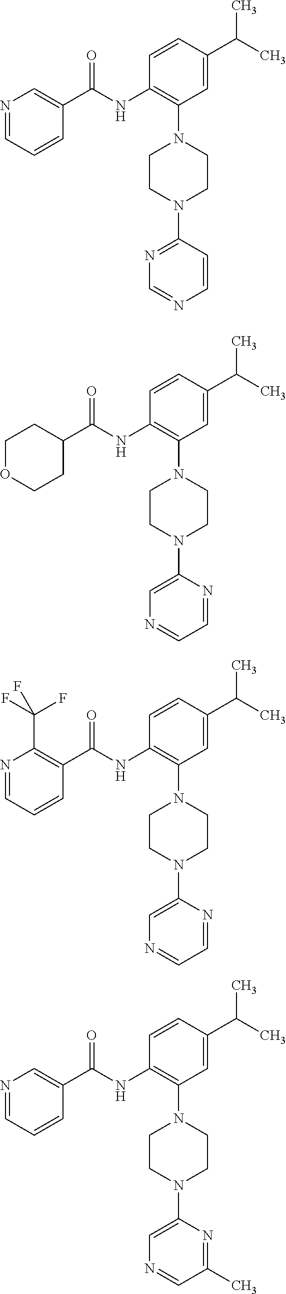 [SMILES: CC(C)c1ccc(NC(=O)c2cccnc2)c(N2CCN(c3ccncn3)CC2)c1 ; CC(C)c1ccc(NC(=O)C2CCOCC2)c(N2CCN(c3cnccn3)CC2)c1 ; CC(C)c1ccc(NC(=O)c2cccnc2C(F)(F)F)c(N2CCN(c3cnccn3)CC2)c1 ; Cc1cncc(N2CCN(c3cc(C(C)C)ccc3NC(=O)c3cccnc3)CC2)n1] 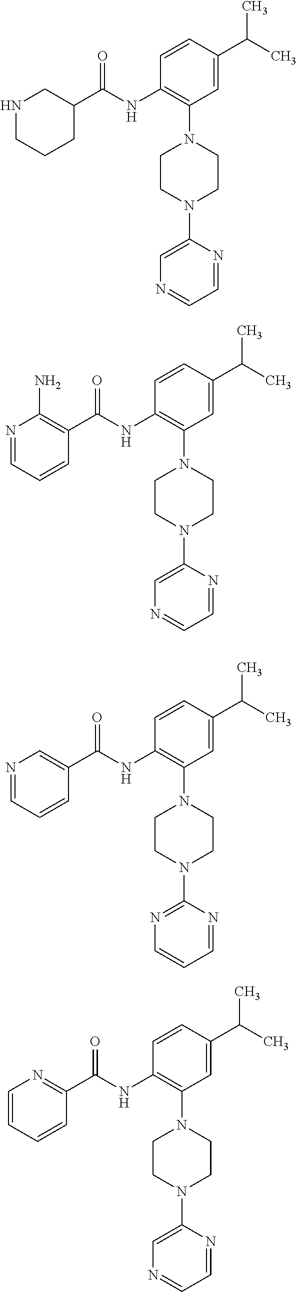 [SMILES: CC(C)c1ccc(NC(=O)C2CCCNC2)c(N2CCN(c3cnccn3)CC2)c1 ; CC(C)c1ccc(NC(=O)c2cccnc2N)c(N2CCN(c3cnccn3)CC2)c1 ; CC(C)c1ccc(NC(=O)c2cccnc2)c(N2CCN(c3ncccn3)CC2)c1 ; CC(C)c1ccc(NC(=O)c2ccccn2)c(N2CCN(c3cnccn3)CC2)c1] 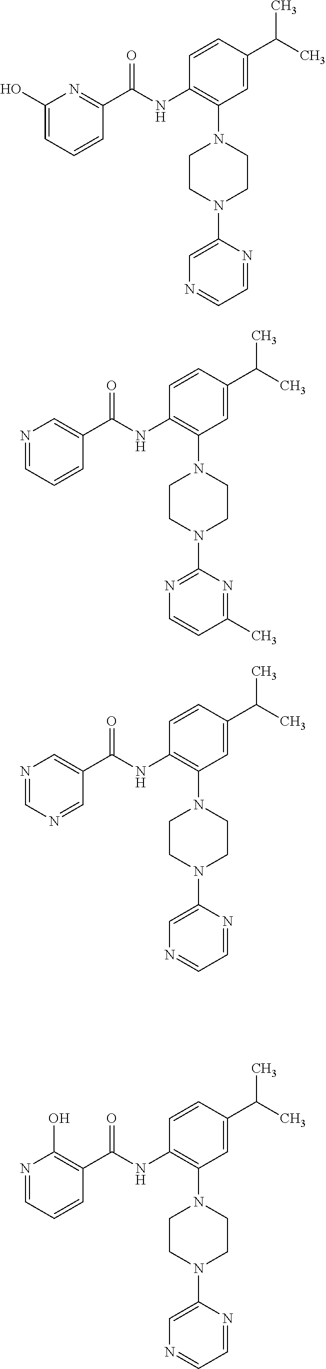 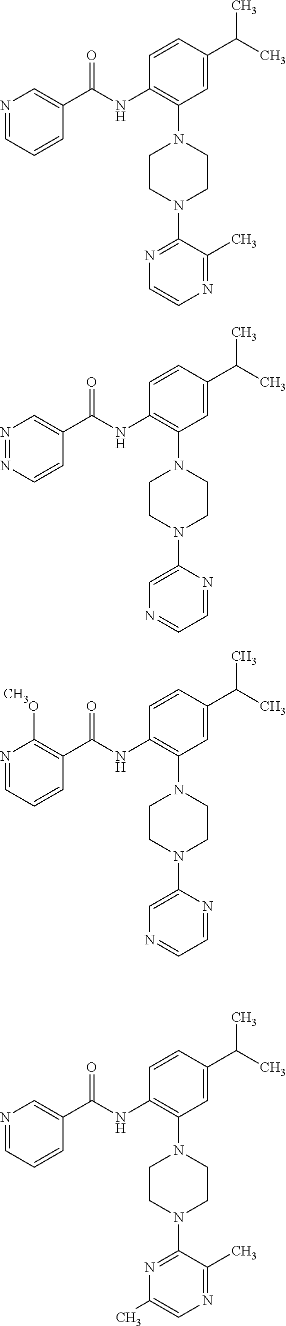 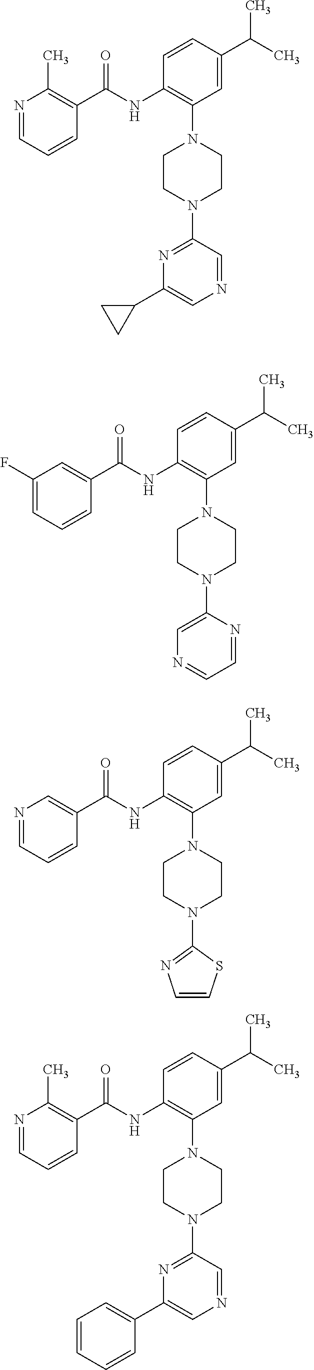 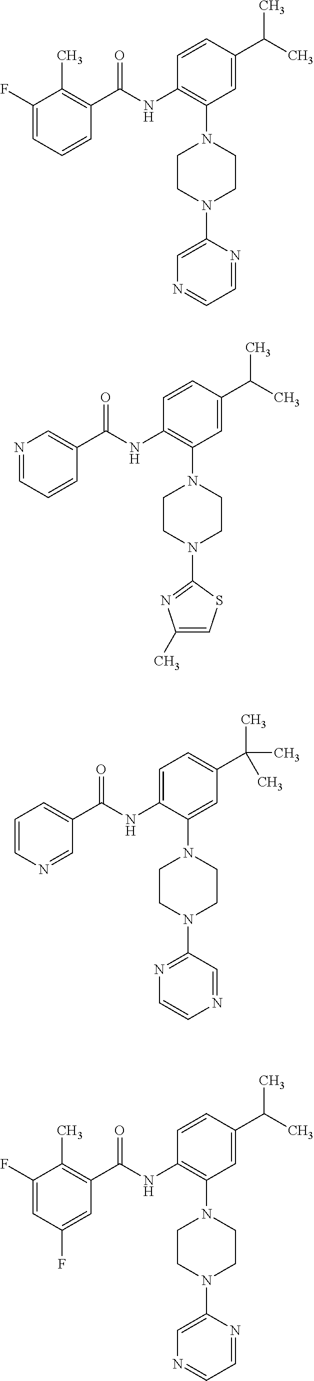 [SMILES: Cc1c(F)cccc1C(=O)Nc1ccc(C(C)C)cc1N1CCN(c2cnccn2)CC1 ; Cc1csc(N2CCN(c3cc(C(C)C)ccc3NC(=O)c3cccnc3)CC2)n1 ; CC(C)(C)c1ccc(NC(=O)c2cccnc2)c(N2CCN(c3cnccn3)CC2)c1 ; Cc1c(F)cc(F)cc1C(=O)Nc1ccc(C(C)C)cc1N1CCN(c2cnccn2)CC1] 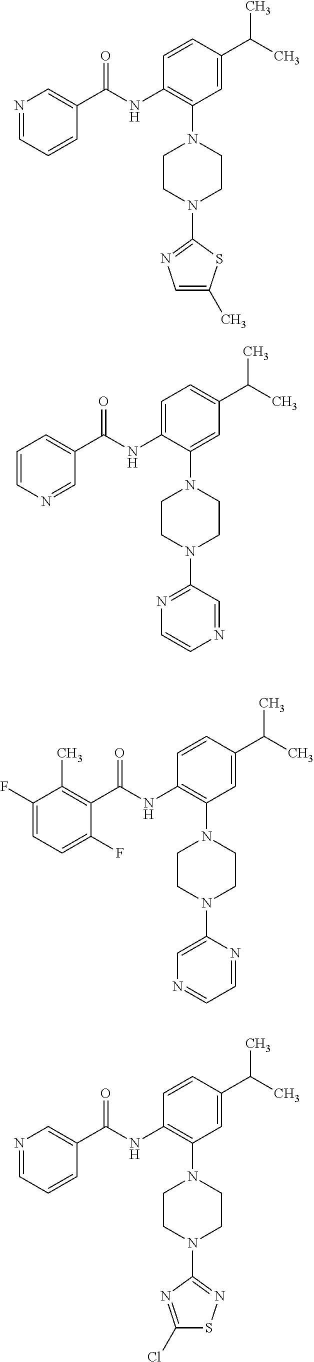 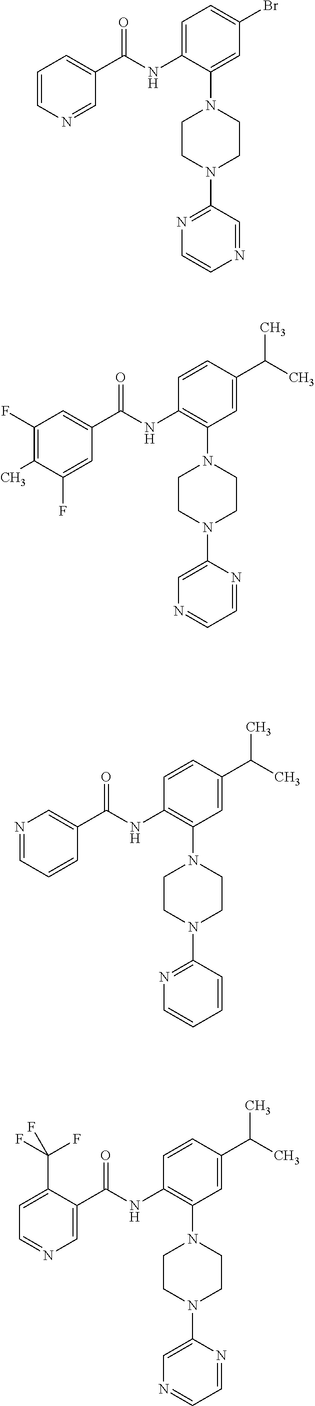 [SMILES: O=C(Nc1ccc(Br)cc1N1CCN(c2cnccn2)CC1)c1cccnc1 ; Cc1c(F)cc(C(=O)Nc2ccc(C(C)C)cc2N2CCN(c3cnccn3)CC2)cc1F ; CC(C)c1ccc(NC(=O)c2cccnc2)c(N2CCN(c3ccccn3)CC2)c1 ; CC(C)c1ccc(NC(=O)c2cnccc2C(F)(F)F)c(N2CCN(c3cnccn3)CC2)c1] 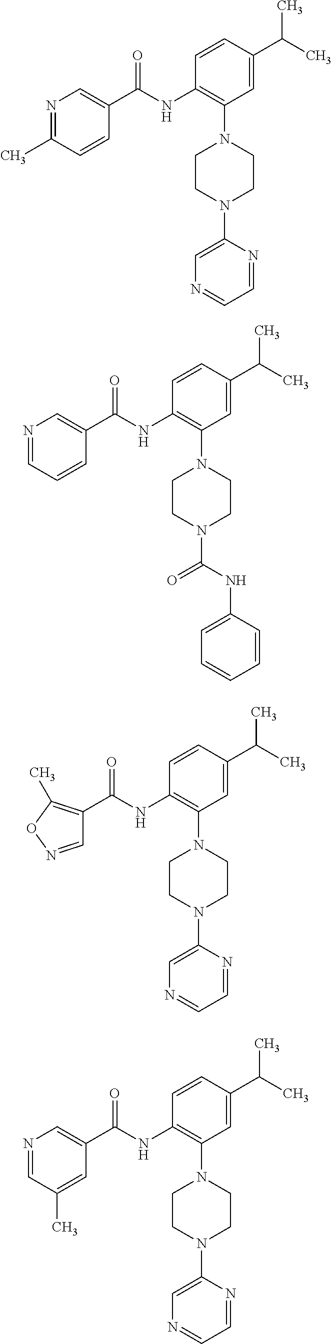 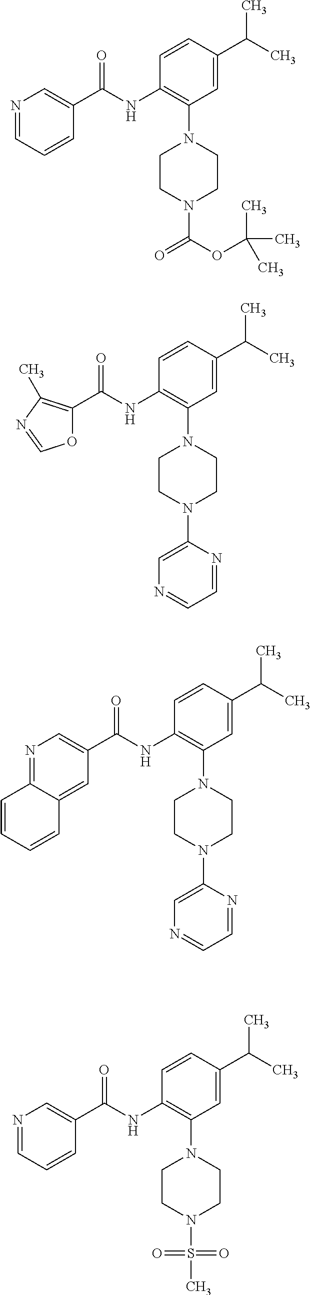 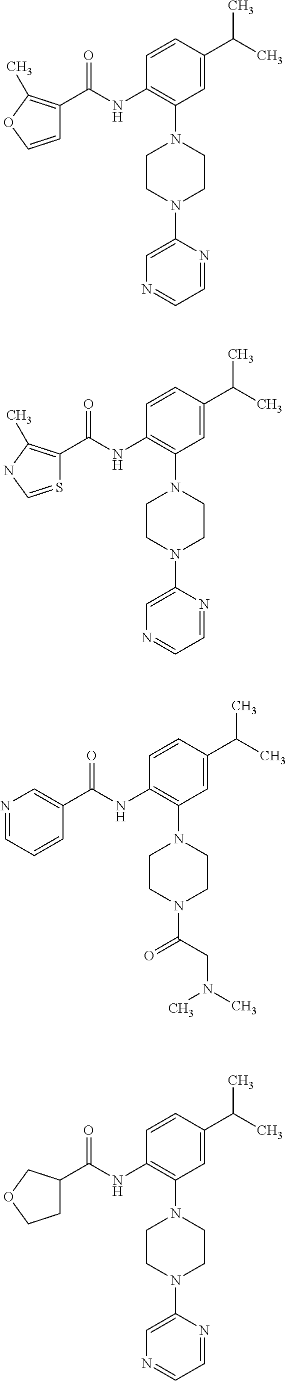 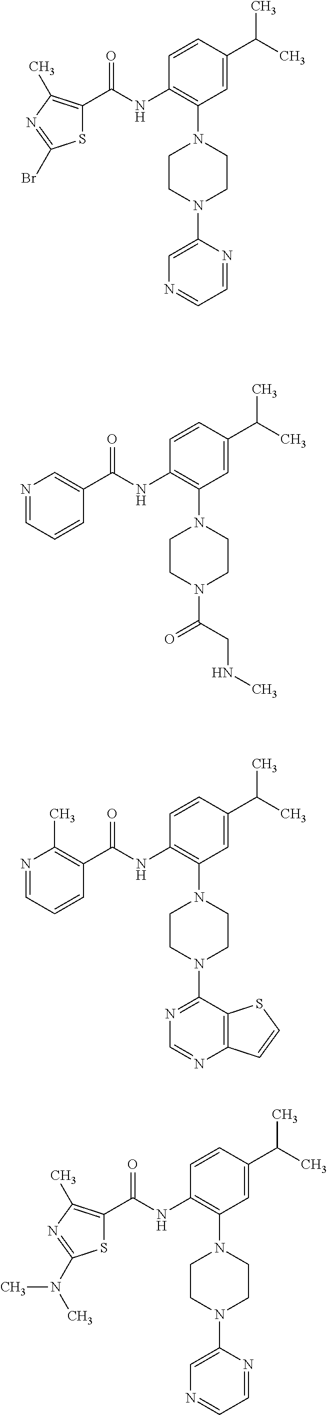 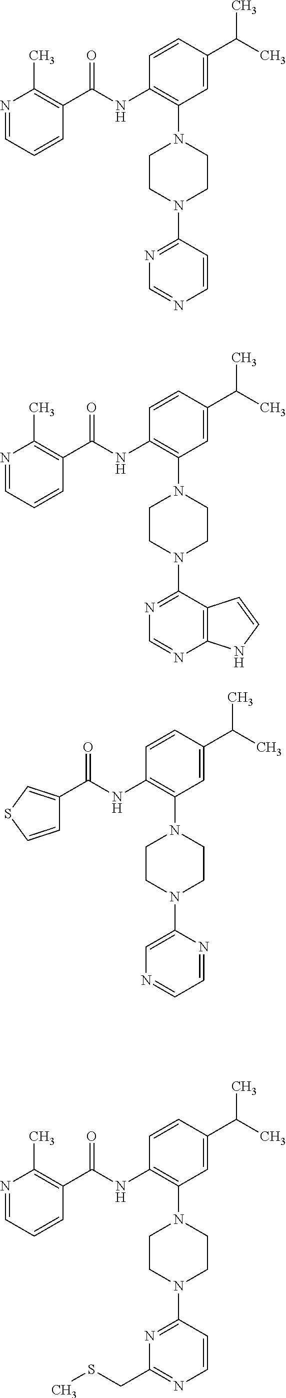 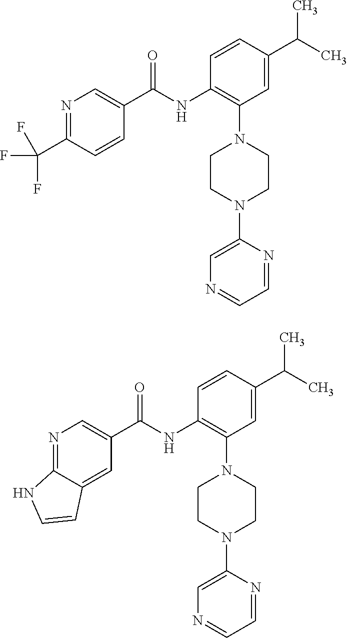 [SMILES: CC(C)c1ccc(NC(=O)c2ccc(C(F)(F)F)nc2)c(N2CCN(c3cnccn3)CC2)c1 ; CC(C)c1ccc(NC(=O)c2cnc3[nH]ccc3c2)c(N2CCN(c3cnccn3)CC2)c1]
or a pharmaceutically acceptable salt thereof.

13. A pharmaceutical composition comprising an effective amount of one or more compounds of claim 1, or a pharmaceutically acceptable salt thereof, and at least one pharmaceutically acceptable carrier.

14. The pharmaceutical composition of claim 13, further comprising at least one additional therapeutic agent, wherein the at least one additional therapeutic agent is not a compound of claim 1 and wherein the at least one additional therapeutic agent is is selected from an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, and an antibody therapy (monoclonal or polyclonal).

15. A method for treating HCV infection in a patient, the method comprising administering to the patient an effective amount of one or more compounds of claim 1, or a pharmaceutically acceptable salt thereof.

16. The method of claim 15, further comprising administering to the patient an effective amount of at least one additional therapeutic agent, wherein the at least one additional therapeutic agent is not a compound of claim 1 and wherein the at least one additional therapeutic agent is selected from an interferon, an immunomodulator, a viral replication inhibitor, an antisense agent, a therapeutic vaccine, a viral polymerase inhibitor, a nucleoside inhibitor, a viral protease inhibitor, a viral helicase inhibitor, a virion production inhibitor, a viral entry inhibitor, a viral assembly inhibitor, and an antibody therapy (monoclonal or polyclonal).
